(19) United States
(12) Patent Application Publication
(10) Pub. No.: US 2009/0181410 A1
(43) Pub. Date: Jul. 16, 2009

(54) IMMUNOGLOBULIN PEPTIDES AGAINST ASIAN PANGASIUS CATFISH

(75) Inventor: Yun-Hwa Peggy Hsieh, Tallahassee, FL (US)

Correspondence Address: JAGTIANI + GUTTAG, 10363-A DEMOCRACY LANE, FAIRFAX, VA, 22030, US

(73) Assignee: FLORIDA STATE UNIVERSITY RESEARCH FOUNDATION, Tallahassee, FL (US)

(21) Appl. No.: 12/233,879

(22) Filed: Sep. 19, 2008

Related U.S. Application Data
(60) Provisional application No. 61/021,246, filed on Jan. 15, 2008.

(51) Int. Cl. G01N 33/53; C12N 5/06; C07K 16/18; G01N 33/543

(52) U.S. Cl. 435/7.21; 435/346; 530/387.1; 530/391.3; 436/518

(57) ABSTRACT

The present invention relates in part to antibodies for the performance of immunoassays to determine whether a test sample, such as a food sample, contains tissue derived from a Pangasius species of fish, such as tra or basa. Such antibodies, either alone or in combination, specifically bind to a thermostable antigen from a Pangasius species. The present invention further relates to methods for conducting immunoassays that may use such antibodies to detect the presence of a test antigen in a test sample that is derived from a Pangasius species of fish, such as tra or basa. In addition, such antibodies may be part of a test kit that may also contain one or more test reagent(s) or items of test equipment.

### CROSS-REFERENCE TO RELATED APPLICATIONS

This application claims the priority date of co-pending. Prov. App. No. 61/021,246, entitled “IMMUNOGLOBULIN PEPTIDES AGAINST ASIAN PANGASIUS CATFISH,” filed Jan. 15, 2008, and the entire disclosure and contents of this provisional application are hereby incorporated by reference.

### GOVERNMENT INTEREST STATEMENT

This invention was made with support from the State of Florida under Florida Department of Health Grant No. 06NIR. The State of Florida may have rights to this invention.

### BACKGROUND

1. Field of the Invention

The present invention broadly relates to immunoglobulin polypeptides or antibodies that recognize an antigen from a Pangasius species in a test sample, such as a food sample, as well as kits containing such immunoglobulin polypeptides or antibodies. The present invention further broadly relates to methods for detecting the presence of a test antigen from a Pangasius species in a test sample, such as a food sample, using such immunoglobulin polypeptides or antibodies.

2. Background of the Invention

Basa (Pangasius bocourti) and tra (Pangasius hypophthalmus) are members of the Pangasiidae family of catfish, which are found throughout most of Southeast Asia. Basa and tra are among the most popular scaleless fish that have been grown by Vietnamese and Cambodian fish farmers in cages along the Mekong Delta for decades. Basa is a tasty white fish with a delicate texture. Compared to basa, tra is faster growing and cheaper to produce, but the eating quality of tra may be considered inferior to basa because of thinner fillets and coarser texture. After the U.S. trade embargo with Vietnam being lifted in 1994, U.S. seafood importers began increasingly to ship basa fillets to the U.S. Lower production costs are believed to be the main reason for the rapid growth in importation of basa fish from this region. U.S. government records reveal that in less than 15 years, Asian catfish imports have risen from about 5 million pounds to over 50 million pounds in 2006. Currently, the U.S. market accounts for 40% of total exports of frozen basa and tra fillets from Asia.

Tra and basa have become the most prominent species substituted for U.S. domestic catfish, grouper and snapper in restaurant-served dishes. Fillets of farm-raised Pangasius fish are usually found to be mislabeled and are marketed in the U.S. as true catfish fillets or wild-caught grouper. Most U.S. importers simply refer to tra as “basa” or simply call it catfish. In fact, a number of importers have created a new brand name, “Cajun Delight Catfish,” to make basa appear as if it were grown on the Mississippi River. See, e.g., http://www.seafoodbusiness.com/buyguide/issue_basa.htm. Such labeling is no longer allowed following the Food and Drug Administration (FDA) ruling that only species from the family Ictaluridae may be sold as true catfish. See Federal Food, Drug, and Cosmetic Act §403(b) stating, “[a] food shall be deemed to be misbranded . . . if it is offered for sale under the name of another food.”

In addition, authorities in the catfish producing states of Mississippi, Louisiana, Arkansas and Georgia have banned Asian catfish because of public health concerns. Aqua-cultured fish from Asian countries are mainly grown in river cages or in ponds by small scale farmers with limited regulations and lax law enforcement to ensure a safe product. The FDA found that imported Asian aquaculture products often contain illegal drugs such as fluoroquinolones, Malachite green dye, nitrofurans, and chloramphenicol that are prohibited in seafood sold in the U.S (http://www.cfsan.fda.gov/˜frf/seadwpe.html#q12). The FDA has a “zero tolerance” policy on these agents because they pose direct and indirect health hazards to humans. In contrast, unapproved compounds have not been found in any domestic aquacultured products such as U.S. domestic catfish or wild caught fish, such as grouper.

Currently, DNA-based PCR assays and protein-based isoelectric focusing (IEF) are available techniques for species identification from a food sample, but both qualitative methods require major laboratory equipment, long assaying time (hours to days), trained analysts to conduct the assay, and authentic fish standards for comparison. PCR techniques may be used for cooked fish tissue; however, they are expensive, require laborious extraction, and are prone to contamination. Furthermore, PCR techniques suffer from a lack of information and databases that may be used for accurate DNA sequence comparison. On the other hand, IEF methods are only suitable for raw fish identification, and factors such as storage condition and closely related species tend to complicate data interpretation.

To discourage the widespread illegal practices of fish species substitution and mislabeling at different trade levels, an economic, reliable and rapid test is urgently needed, especially for the identification of Pangasius fish species, such as tra and/or basa.

### SUMMARY

According to a first broad aspect of the present invention, hybridoma cell lines deposited as one of ATCC Nos. ______, is provided along with antibodies, comprising T7E10, T1G11, F7B8, or F1G11, produced by such hybridoma cell lines, or a fragment(s) or a portion(s) thereof, as well as test kits including such antibodies.

According to a second broad aspect of the present invention, a method is provided comprising the following steps: (a) combining a primary detection antibody and the contents of a test sample; and (b) determining whether the primary detection antibody binds to a test antigen that may be present in the test sample, wherein the test antigen is from a Pangasius species, wherein the primary detection antibody specifically binds to one or more epitopes on the test antigen, and wherein the one or more epitopes of the test antigen are not present in a non-Pangasius species.

According to a third broad aspect of the present invention, a method is provided comprising the following steps: (a) combining the contents of a test sample with a capture antibody; and (b) determining whether the capture antibody binds to a competing antigen, wherein the capture antibody binds to both the competing antigen and a test antigen from a Pangasius species that may be present in the test sample, and wherein the capture antibody is immobilized on or to a solid phase material.

According to a fourth broad aspect of the present invention, a method is provided comprising the following steps: (a) combining a primary detection antibody with the contents of a sample; and (b) determining whether the primary detection antibody binds to a test antigen present in the sample, wherein the test antigen is a tropomyosin protein from a Pangasius species, wherein the primary detection antibody specifically binds to one or more epitopes on the tropomyosin protein from a Pangasius species, and wherein the one or more epitopes on the tropomyosin protein from a Pangasius species are not present on a tropomyosin protein from a non-Pangasius species.

### BRIEF DESCRIPTION OF THE DRAWINGS

FIGS. 1 and 1-1 together represent a table showing the immunoreactivity of MAbs T7E10, T1G11 F7B8 and F1G11 against cooked fish and non-fish samples as determined by indirect non-competitive ELISA with absorbance readings measured at 410 nm, wherein the levels of immunoreactivity are categorized by their absorbance readings as follows: <0.15=“—”; 0.15-0.199=“±”; 0.2-0.499=“+”; 0.5-0.999=“++”; >1=“+++”;

FIG. 2 is a bar graph showing the binding of MAb T7E10 to cooked sample extracts from various fish and animal species by indirect ELISA measured at an absorbance of 410 nm with strong immunoreactivity demonstrated for T7E10 to cooked (c) tra and basa extracts without cross reactivity with other non-Pangasius fish or animal samples, wherein BA=basa; T=tra; RG=red grouper; GG=gag grouper; YEG=yellow edge grouper; BGC=black grouper (carbo); S=scamp; CF=channel catfish; CG=camouflage grouper; CT=coral trout; AJ=amberjack; FS=farm salmon; CO=cobia; AC=atlantic croaker; YT=yellowfin tuna; RS=red snapper; TIL=tilapia; DTG=dusky tail grouper; OSG=orange spotted grouper; RMG=red mouth grouper; SG=squaretail grouper; TH=tomato hind; Cb=chicken breast; CT=chicken thigh; Tb=turkey breast; Tt=turkey thigh; P=pork; B=beef; R=rabbit; E=elk; and H=horse;

FIG. 3 is an image of a Western blot of cooked fish extracts from various species probed with MAb F7B8 revealing an ˜36 kDa antigenic protein band for all fish species, wherein TU=tuna; T=tra; B=basa; CF=catfish; SWF=swordfish; YEG=yellow edge grouper; WS=wild salmon; AJ=amberjack; and AH=alaskan halibut; STD=molecular weight standards;

FIG. 4 is an image of a Western blot of cooked fish extracts from various species probed with MAb F1G11 revealing an ˜36 kDa antigenic protein in extracts from all fish species, wherein T=tra; B=basa; RS=red snapper; GS=gray snapper; RG=red grouper; GG=gag grouper; and CF=catfish; STD=molecular weight standards;

FIG. 5 is an image of a Western blot of cooked fish extracts from various species probed with MAb T7E10 revealing an approximately 36 kDa (major) and 75 kDa (minor) antigenic protein bands that are only present in extracts from tra and basa extracts and not with extracts from other fish species, wherein S=scamp; T=tra; CF=catfish; RG=red grouper; YEG=yellow edge grouper; GG=gag grouper; and BG=black grouper; STD=molecular weight standards;

FIG. 6 is an image of a Western blot of cooked fish extracts from various species probed with MAb T1G11 revealing two antigenic protein bands between 15 and 20 kDa in a cooked tra extract and four antigenic protein bands between 13 and 18 kDa in a cooked basa extract, wherein T=tra; B=basa; RS=red snapper; GS=gray snapper; RG=red grouper; GG=gag grouper; and CF=catfish; STD=molecular weight standards;

FIG. 7 is a summary table providing the sizes of antigenic proteins in cooked tra and basa fish extracts revealed by Western blot analysis using T7E10, T1G11, F7B8 and F1G11 as primary detection antibodies;

FIG. 8 is a table providing optical density (O.D.) measurements at 410 nm in two ELISA replicate experiments using an extract from cooked tra extract containing 0.5 μg/100 μL of soluble proteins with average and standard deviation (S.D.) values for different antibody combinations to allow for epitope comparison by measuring the Additivity Index (A.I.) for each pair-wise combination of MAbs T7E10, T1G11, F7B8 and F1G11;

FIG. 9 is a bar graph showing absorbance readings at 410 nm for ELISA experiments using MAbs F7B8 and T7E10 alone or together using an extract from cooked tra extract containing 0.5 μg/100 μL of soluble proteins to show increased detection of antigen with pair-wise combination of F7B8 and T7E10 antibodies in 1:1 ratio, wherein BLK=blank;

FIG. 10 is a bar graph showing absorbance at 410 nm for optimized ELISA experiments using MAb F7B8 as the capture antibody and biotinylated MAb T7E10 as the primary detection antibody for extracts from a cooked basa sample using different extraction buffers (0.15M NaCl, 0.5M NaCl, 0.15M KCl, 0.5M KCl, and water); and

FIG. 11 is a bar graph showing the specificity of the developed ELISA for basa and tra antigens by measuring absorbance at 410 nm for extracts from various fish and non-fish species analyzed by the sandwich ELISA using purified MAb F7B8 as the capture antibody and biotin-conjugated MAb T7E10 as the primary detection antibody.

### DETAILED DESCRIPTION

### DEFINITIONS

For purposes of the present invention, the term “antigen” refers to a macromolecule or complex, such as a protein, polysaccharide, etc., that is specifically bound by an antibody or immunoglobulin-like polypeptides, or a portion or fragment thereof. For example, such antigen may be a molecule present in a tissue sample derived from a Pangasius species, such as tra or basa. The term “antigen” may also refer to a molecule or substance that is used as an immunogen injected into an animal to cause the production of antibodies that specifically bind to the antigen.

For purposes of the present invention, the term “epitope” refers to a region of a macromolecule that is bound by an antibody or immunoglobulin-like polypeptides, or a portion or fragment thereof. The term “epitope” may also be referred to as an antigenic determinant.

For purposes of the present invention, the term “antibody” refers to one or more immunoglobulin or immunoglobulin-like polypeptide(s), or portion(s) or fragment(s) thereof, that retain the ability to bind to an antigen. Such antibody may include a complex of immunoglobulin or immunoglobulin-like polypeptide(s), which may be linked together by one or more disulfide linkages, or a single immunoglobulin or immunoglobulin-like polypeptide, such as scFv, with each immunoglobulin or immunoglobulin-like polypeptide having at least a portion of one or more immunoglobulin domain(s). Such immunoglobulin domain(s) will generally include at least a portion of a variable region or domain responsible for specific binding to an antigen. In general, such antibody will include at least a portion of a heavy chain and at least a portion of a light chain, which may be linked together to form a single chain. Such antibody may include polyclonal or monoclonal antibodies. Such antibody may include a full sized antibody of any type (e.g., IgG, IgM, IgE, IgD, etc.) or a fragment of an antibody, such as Fab or F(ab′)₂. Such antibody may include any immunoglobulin or immunoglobulin-like polypeptide, or fragment thereof, engineered by recombinant techniques to have desired binding properties or other characteristics, and such antibody may include any antibody encoded by a polynucleotide sequence that has been subjected to mutagenesis. Further, such antibody may be engineered to have or include an assayable tag or label.

For purposes of the present invention, the term “immunoglobulin-like polypeptide” refers to any portion, fragment, or engineered version of an antibody or immunoglobulin polypeptide having at least a portion of one or more immunoglobulin domains including at least a portion of the variable region or domain of the heavy and/or light chains. Such immunoglobulin-like polypeptide will generally retain the ability to bind to an antigen either alone or in association with other immunoglobulin or immunoglobulin-like domains. In general, such immunoglobulin-like polypeptide may include any protein that has been modified by digestion, chemical treatment, recombinant techniques, etc. from its natural or original state. For example, such immunoglobulin-like polypeptide may include a Fab or F(ab′)₂ fragments or a scFv.

For purposes of the present invention, the term “Pangasius species” refers to any species of fish classified within the Pangasiidae family of fish species, such as, for example, tra (Pangasius hypophthalmus) or basa (Pangasius bocourti). Conversely, the term “non-Pangasius species” refers to any species of fish that is not classified within the Pangasiidae family of fish species, such as, for example, species from the Ictaluridae family of catfish.

For purposes of the present invention, the term “assayable tag or label” refers to a chemical group that is chemically or covalently linked or bonded to a component of an immunoassay, such as a primary or secondary detection antibody or a competing antigen as the case may be, or otherwise stably associated, such as by intermolecular forces, to such component. Such “assayable tag or label” may include any chemical entity, such as an enzyme, radionuclide, fluorophores, etc. as further described herein, that are capable of being detected in an immunoassay.

For purposes of the present invention, the term “immunoassay” refers to any technique or method known in the art that may be used to detect the presence of a test antigen in a test sample based on the ability of an antibody to bind to an antigen.

For purposes of the present invention, the terms “solid phase” or “solid phase material” refer interchangeably to any material that may be used to immobilize one or more components of an immunoassay to facilitate their separation, isolation, and/or detection from all other reaction components and sample contents. For example, such solid phase materials may be used to directly or indirectly immobilize any reaction component and/or sample contents, such as an antibody, immunoglobulin-like polypeptides, or portion(s) or fragment(s) thereof, a test antigen, and/or a competing antigen.

For purposes of the present invention, the term “test sample” generally refers to a food or other sample that may be tested for the presence of a test antigen. Such test sample may include any tissue source, such as, for example, a food or agricultural product, or any crude or at least partially purified extract derived from such tissue source. For example, such test sample may be any tissue source or product intended for human or animal consumption, or an extract prepared therefrom.

For purposes of the present invention, the terms “test antigen” or “antigen of interest” refer interchangeably to an antigen present in tissue from a Pangasius species of fish, such as, for example, tra or basa, that may be targeted for direct or indirect detection using any immunoassay format described herein. For example, such test antigen or antigen of interest may refer to an antigen that is present only in tissue extracts derived from a Pangasius species, such as, for example, tra or basa. Alternatively, for example, such test antigen or antigen of interest may refer to an antigen that is present in tissue extracts derived from a Pangasius species, such as, for example, tra or basa, and not substantially present in all other relevant species of fish.

For purposes of the present invention, the term “thermostable” refers to a macromolecule or antigen, such as a protein, etc., that remains soluble at high temperatures. For example, the term “thermostable” may refer to a protein antigen that remains in solution after heating a sample containing such antigen at approximately 100° C. for about 15 min.

For purposes of the present invention, the term “competing antigen” refers to an antigen which competes with the test antigen for binding to a primary detection antibody or a capture antibody. For example, such competing antigen may be a protein that is identical or similar to, or a fragment of, the test antigen. Such competing antigen may be may be purified or synthesized in vitro, and/or such antigen may be linked to an assayable tag or label.

For purposes of the present invention, the term “blocking agent” generally refers to any protein or molecule that may be used to coat the surface of a solid phase material after an intended reaction component, such as a capture antibody or competing antigen, has been bound to the surface of a solid phase material to discourage or block the ability of other reaction components to bind non-specifically to the surface of the solid phase material. For example, such blocking agent may include bovine serum albumin (BSA), gelatin, non-fat dry milk, etc.

For purposes of the present invention, the term “specifically” in reference to the binding interaction of an antibody and an antigen from a particular species, such as an antigen from a Pangasius species including tra or basa, may refer to the ability of the antibody to selectively bind to such antigen with high affinity or avidity. The term “specifically” may also refer to the ability of the antibody to selectively bind to such antigen to a much greater extent than other antigens or similar antigens from a different species, such as a non-Pangasius species.

For purposes of the present invention, the terms “test equipment” or “item of test equipment” generally refer to equipment which may be included in a test kit. Such test equipment item may include, but is not limited to, any solid phase materials, such as carriers, substrates, containers, vials, tubes, dipsticks, beads, etc., and/or any other materials, such as filters, bags, labels, instructional materials (e.g., written materials or on electronic storage media), etc. Test equipment may have associated therewith primary or secondary detection antibodies, capture antibodies, test reagent(s), etc.

For purposes of the present invention, the terms “test reagent” or “test reagent(s)” refer interchangeably to reagents, other than a primary detection antibody and/or a capture antibody, which may be included in a test kit. Such “test reagent(s)” may include, but are not limited to, a secondary detection antibody, a second capture antibody, a competing antigen, solutions or buffers, materials for different assays, standards, nucleic acid constructs, etc.

For purposes of the present invention, when comparing two or more different antibodies, the phrase “similar chemical structure” in reference to such antibodies may refer to the similarity in primary amino acid sequence of the variable domains of the heavy and/or light chains of such antibodies. For example, such antibodies may be considered to have a similar chemical structure if the primary amino acid sequence of the variable domains of the heavy and light chains of such antibodies is at least 80% identical, or such antibodies may be considered to have a similar chemical structure, for example, if the primary amino acid sequence of the variable domains of the heavy and light chains of such antibodies is at least 90% or 95% identical. Two or more antibodies may also be considered to have a “similar chemical structure” if such antibodies share binding strength, affinity, or avidity for a particular antigen. For example, such antibodies may considered to have a “similar chemical structure” if each of such antibodies has a binding affinity or avidity for a particular antigen that does not vary by more than 10% compared to all other such antibodies or if each of such antibodies has a binding affinity or avidity for a particular antigen that does not vary by more than 5% compared to all other such antibodies. In addition, two or more antibodies may be considered to have a “similar chemical structure” if such antibodies bind specifically to the same epitope(s) of a particular antigen, which may be defined in terms of a primary amino acid sequence of the antigen that is specifically bound by such antibodies or in terms of a conformational shape or domain of the antigen that is specifically bound by such antibodies.

### Description

At the present time, there is believed to be no rapid immunoassay commercially available to identify the fish species of origin for a food sample. Monoclonal antibodies have the potential to specifically bind and detect epitopes of proteins that derive from a particular species or family of related species. Immunoassays based on the use of monoclonal antibodies may be inexpensive and carried out rapidly (e.g., complete in minutes) even by untrained persons, such as inspectors or fisherman, to determine and/or verify the true identity of the fish species of origin for a particular food sample. Furthermore, immunoassays based on the use of monoclonal antibodies may be used to bind and detect thermostable proteins that are unique to a particular fish species or family of related fish species, thereby allowing the determination of the species of origin for a food sample regardless of whether the food sample is raw, frozen, cooked, or canned.

Efforts to develop antibodies specific for antigens from a particular fish species have been difficult to achieve. Although a few monoclonal antibodies have been developed for species identification purposes in fish, many of these antibodies may cross-react with fish species other than the fish species of interest. For example, two monoclonal antibodies, C1C1 and C2A2, have been reported for identification of Red Snapper. See, Huang et al., “Development of monoclonal antibodies for red snapper (Lutjanus campechanus) identification using enzyme-linked immunosorbet assay,” J. Agric Food Chem 43:2301-2307 (1995). However, both of these antibodies were found to cross-react with four other snapper species even when both antibodies were used together for detection. Another group reported the development of monoclonal antibody, 3D12 and 1A4, for the identification of raw grouper fillets. See, Asensio et al., “Development of a monoclonal antibody for grouper (Epinephelus guaza) identification using indirect enzyme-linked immunosrobent assay.” J Food Prot 66:886-889 (2003); and Asensio et al., “Development of a monoclonal antibody for grouper (Epinephelus guaza) and wreck fish (Polyprion americanus) authentication using an indirect ELISA.” J Food Sci 68:1900-1903 (2003). Although the 1A4 antibody had the ability to identify grouper in cooked products, it also cross-reacted with a wreck fish sample. However, no polyclonal or monoclonal antibodies that specifically bind to Asian Pangasius fish species are known to have been developed.

As described further herein, some embodiments of the present invention may provide for the identification of thermostable antigens present in tissue extract samples which may be derived from a Pangasius species, such as tra or basa, using monoclonal antibodies. In addition, some embodiments of the present invention provide immunoassays to detect the presence of tissue which may be derived from a Pangasius species of fish, such as tra or basa, in a test sample. Newly identified monoclonal antibodies (mAbs) are also provided herein that bind to thermostable antigens present in an extract sample from a Pangasius species, such as tra or basa. Indeed, two of the four monoclonal antibodies identified herein, 7E10.D8.E6 (T7E10) and 1G11.D3.E2 (T1G11), are shown to bind with specificity and affinity to one or more thermostable antigens present only in extracts from a Pangasius species. The other two monoclonal antibodies identified herein, 1G11.D3.D12 (F1G11) and 7B8.G11.F1 (F7B8), bind to one or more antigens in extract samples taken from a Pangasius species, including tra and/or basa, but may also cross-react with the same or similar antigens from other fish species. All four of these antibodies may be used to detect such antigens in both raw and cooked samples of tissue extracts which may be taken from a Pangasius species, including tra and/or basa.

According to some embodiments of the present invention, these newly identified antibodies and others directed specifically against antigens from a Pangasius species may be used alone or in combination to construct a wide variety of immunoassays to detect the presence of one or more antigens in a test sample that would only be present in extracts taken at least in part from tissue derived from a Pangasius species, such as tra or basa. In other words, immunoassays based on these and other antibodies may be used to specifically detect the presence of tissue derived from a Pangasius species, such as tra or basa, in a food sample or other materials, and which may be used for inspection, forensic, scientific, agricultural, or other purposes.

According to some embodiments of the present invention, the types of samples that may be tested for the presence of tissue from a Pangasius species, such as tra and/or basa, may include crude or at least partially purified extract samples taken from a relevant source, such as a food or agricultural source or product. Such food sources may include frozen, partially frozen, thawed, raw, and/or cooked meat from one or more animals. For example, such food source may include fish fillets, ground and/or processed meats, etc. Such food source may be intended for human and/or animal consumption or for some other industrial, scientific, or agricultural purpose. According to some embodiments, the food source may be a fish fillet marketed as being grouper, snapper, catfish, etc. In addition, such food source may include surimi samples, which are used to prepare imitation seafood, and particularly imitation shellfish. According to other embodiments, the food source may be unknown or merely suspected to be of a certain type and origin.

According to some embodiments, a raw (i.e., uncooked) food or agricultural source or product may be heated to compare the results of an immunoassay conducted on extract samples taken from both raw and/or cooked sources or products. Alternatively, an extract sample taken from a raw source or product may be heated and optionally compared to an unheated extract sample taken from the raw food source. For example, an agricultural or food product, or extract sample derived therefrom, may be heated in boiling water for about 15 minutes, or by autoclaving for about 15 minutes at 121° C. at 1.2 bars of pressure.

According to some embodiments, a frozen, partially frozen, thawed, raw, and/or cooked food or agricultural source or product may be mechanically mashed or pulverized to break up the food source and release cellular contents present in tissues of the food source into an extraction buffer. After homogenization, the extract may be centrifuged, and the supernatant (crude extract) removed and tested according to some embodiments of immunoassays of the present invention. Alternatively, a crude extract obtained from a food source may be further purified to concentrate antigens that may be present in the test sample.

### Immunoassays

According to some embodiments of the present invention, a wide variety of immunoassays may be used to detect the presence of an antigen of interest or test antigen from a Pangasius species, such as tra and/or basa, in a test sample. Such immunoassays may include, for example, precipitation and agglutination techniques, heterogeneous and homogeneous immunoassays, enzyme immunoassays (EAs) such as enzyme-linked immunosorbent assays (ELISAs), fluorescent immunoassays, radioimmunoassay, Western blots, immunosensors, lateral flow tests, immunohistochemical assays, immunoarrays, other biochemical techniques, etc. With the exception of certain biochemical techniques, including precipitation and agglutination, these immunoassays generally may depend on the use of an assayable tag or label that may be used for detection and measurement of an antibody or competing antigen to determine whether a test antigen may be present in a test sample.

Many immunoassays or ELISAs included in some embodiments of the present invention may be grouped into two broad categories of homogeneous or heterogeneous immunoassays. In general, heterogeneous immunoassays or ELISAs rely on the use of a solid phase material to immobilize an antigen or antibody to its surface to allow washing and removal of unbound immunoassay components in a stepwise fashion. In contrast, homogeneous immunoassays may be performed entirely in solution and may not require removal of immunoassay components or washing steps. For example, a homogeneous immunoassay for the detection of an antigen of interest in a test sample according to some embodiments of the present invention may rely on the use of a competing antigen directly linked to an assayable tag or label that is capable of binding to a detection antibody that also can bind to the antigen of interest. However, what distinguishes this homogeneous immunoassay from other competitive heterogeneous immunoassays is that the assayable tag or label may be positioned on the competing antigen such that when the competing antigen is bound by the detection antibody, the assayable tag or label is shielded so that the detection of the tagged or labeled competing antigen becomes diminished or eliminated. Therefore, in contrast to heterogeneous competition or inhibition immunoassays, if a test sample contains the antigen of interest, then a greater amount of assayable tag or label will be detected in the presence of the test sample since the antigen of interest binds to the detection antibody in place or instead of the competing antigen, thus allowing for greater amounts of the assayable tag or label to be displayed by free (unbound) competing antigen.

### Heterogeneous Immunoassays and ELISAs

According to embodiments of the present invention, most immunoassays that may be performed are heterogeneous immunoassays that generally rely on the use of some type of solid phase material to immobilize immunoassay components. According to some embodiments of present methods, an immunoassay, such as an enzyme-linked immunosorbent assay (ELISA) or enzyme immunoassay (EIA), may be performed to detect the presence of a test antigen, i.e., an antigen from a Pangasius species, such as tra or basa, in a test sample. Such immunoassay or ELISA technique may employ a variety of approaches known in the art to detect the antigen. For example, a “direct,” “indirect,” or “sandwich” immunoassay or ELISA may be used, which may each further comprise an “inhibition” or “competition” assay approach. In addition, a direct or indirect sandwich immunoassay or ELISA may also be used.

According to some embodiments, a direct immunoassay or ELISA approach generally involves immobilization of a test antigen onto the surface of a solid phase material (or via a capture antibody in the case of a sandwich assay) to allow detection of the test antigen by a primary detection antibody that is directly linked to an assayable tag or label. Such assayable tag or label may include any chemical group known in the art that is capable of detection, and which may be chemically, covalently, or intermolecularly attached, linked, or bonded to the primary detection antibody, as described further below. If a test sample contains the test antigen, then the assayable tag or label may be detected after washing as a result of the primary detection antibody being immobilized on or to the solid phase material via the antigen. Conversely, if a test sample does not contain the test antigen, then the assayable tag or label will not be detected since it will be washed away in the absence of the test antigen immobilized on or to the surface of the solid phase material. The direct immunoassay or ELISA approach has the advantage of being simple and requiring relatively fewer reagents.

According to some embodiments, a direct immunoassay or ELISA approach may include, for example, the following steps. Proteins contained in a crude or at least partially purified extract sample taken from a food or agricultural source or product to be tested may be attached to the surface of a solid phase material by passive adsorption. For example, such crude or at least partially purified extract sample may be present in a coating buffer, such as a carbonate buffer, pH 9.6; Tris-HCl, pH 8.5; or phosphate-buffered saline (PBS), pH 7.2. For example, the contents of the test sample may then be incubated with the solid phase material for about 1 to about 4 hours at 37° C. or overnight at 4° C. Contents of the test sample that do not bind to the surface of the solid phase material may be removed by a repeated wash steps. Then, a primary detection antibody directly linked to an assayable tag or label may be added to bind to any test antigen, i.e., an antigen from a Pangasius species, which may be bound on or to the surface of the solid phase material as a result of being present in the test sample. The primary detection antibody may be added to the solid phase material at appropriate dilution in a blocking buffer that contains a blocking agent for binding on or to exposed surfaces of the solid phase material that are not occupied by contents of the test sample to avoid non-specific binding of the primary detection antibody on or to the solid phase material. In addition, a blocking agent or buffer may be introduced prior to the addition of the primary detection antibody. Once the primary detection antibody has been allowed to bind to any test antigen that may be immobilized on or to the surface of the solid phase material, any unbound antibody may be removed by repeated wash steps. Finally, the presence of the assayable tag or label may be detected by an appropriate assay to indicate whether any test antigen is immobilized on or to the surface of the solid phase material as a result of the test antigen being present in the test sample derived from the food or agricultural source or product.

According to some embodiments, an indirect immunoassay or ELISA approach generally involves immobilization of a test antigen on or to the surface of a solid phase material (or via a capture antibody in the case of a sandwich assay) similarly to the direct immunoassay or ELISA approach described above. However, instead of providing a primary detection antibody that is directly linked to an assayable tag or label, a secondary detection antibody that recognizes and binds to the primary detection antibody is used instead to provide the assayable tag or label. The indirect immunoassay or ELISA approach has the advantage of flexibility in choosing from numerous secondary detection antibodies, such as anti-species antibodies, that are directly linked to an assayable tag or label, many of which are commercially available. In addition, the indirect immunoassay or ELISA approach allows the primary detection antibody to be chosen for its ability to bind the test antigen without complications or effects that may be caused by adding or conjugating an assayable tag or label directly to the primary detection antibody.

According to some embodiments, an indirect immunoassay or ELISA approach may include, for example, the following steps. Proteins contained in a crude or at least partially purified extract sample taken from a food or agricultural source or product to be tested may be attached on or to the surface of a solid phase material by passive adsorption. For example, such crude or at least partially purified extract sample may be present in a coating buffer, such as a carbonate buffer, pH 9.6; Tris-HCl, pH 8.5; or phosphate-buffered saline (PBS), pH 7.2. The contents of the sample may then be incubated with the solid phase material for about 1 to about 4 hours at 37° C. or overnight at 4° C. Contents of the test sample that do not bind on or to the surface of the solid phase material may be removed by repeated wash steps. Then, a primary detection antibody may be added to bind to any test antigen, such as an antigen from a Pangasius species, which may be bound on or to the surface of the solid phase material as a result of being present in the test sample. The primary detection antibody may be added to the solid phase material at appropriate dilution in a blocking buffer that contains a blocking agent for binding on or to exposed surfaces of the solid phase material that are not occupied by contents of the sample to avoid non-specific binding of the primary detection antibody to the solid phase material. In addition, a blocking agent or buffer may be introduced prior to the addition of the primary detection antibody. For example, the primary detection antibody may be incubated with the solid phase material for about 1 hour at room temperature or 37° C.

Once the primary detection antibody has been allowed to bind to any test antigen that may be immobilized on or to the surface of the solid phase material, any unbound antibody may be removed by repeated wash steps. In contrast to the direct approach, a secondary detection antibody directly linked to an assayable tag or label may then be introduced to bind to any primary detection antibody that may be bound to any test antigen immobilized on or to the surface of the solid phase material. The secondary detection antibody may be added to the solid phase material at appropriate dilution in a blocking buffer that contains a blocking agent. For example, the secondary detection may be added to the solid phase material for about 1 hour at room temperature or 37° C. Once the secondary detection antibody has been allowed to indirectly bind to any test antigen that may be immobilized on or to the surface of the solid phase material, any unbound antibody may be removed by repeated wash steps. Finally, the presence of the assayable tag or label may be detected by an appropriate assay to indicate whether any test antigen is immobilized on or to the surface of the solid phase material as a result of the test antigen being present in the test sample derived from the food or agricultural source or product.

According to embodiments of present methods, a sandwich or capture immunoassay or ELISA is generally similar to the direct or indirect methods described above except that the test antigen is not directly bound on or to the surface of the solid phase material. Instead, an additional capture antibody which may be immobilized on or to the surface of the solid phase material is used to bind any test antigen that may be present in a test sample taken from a food or agricultural source or product. With a direct sandwich assay, any test antigen bound by the capture antibody immobilized on or to the solid phase material is detected using a primary detection antibody directly linked to an assayable tag or label that also binds with specificity to the test antigen. With an indirect sandwich assay, any test antigen bound by the capture antibody immobilized on or to the solid phase material may be detected using a primary detection antibody having specificity for the test antigen in combination with a secondary detection antibody directly linked to an assayable tag or label that binds to the primary detection antibody. Whether a direct or indirect sandwich assay approach is used, the presence of an assayable tag or label may be detected by an appropriate assay to indicate whether any test antigen is bound by the capture antibody immobilized on or to the solid phase material as a result of the test antigen being present in the test sample derived from the food or agricultural source or product.

Sandwich immunoassay or ELISA approaches may have the potential advantage of greater specificity for a test antigen. By using two different antibodies for the same test antigen, greater selectivity and sensitivity for the test antigen may be achieved, thus reducing background and non-specific effects. In other words, the assayable tag or label will be present only when both the capture antibody and the primary detection antibody are able to recognize and bind to the test antigen. On the other hand, sandwich assays may have the disadvantage of requiring two or more antibodies (i.e., “matched pairs”) that are capable of binding to sufficiently distinct epitopes on the same test antigen. Further, using a capture antibody and a primary detection antibody from the same species may possibly result in cross-reactivity with an indirect sandwich assay since the secondary detection antibody may bind to both the capture antibody and the primary detection antibody.

According to some embodiments, a sandwich immunoassay or ELISA approach may include, for example, the following steps. A capture antibody that is capable of specifically binding to a test antigen is first immobilized on or to the surface of a solid phase material. For example, the capture antibody may be passively adsorbed onto the surface of the solid phase material in a coating or adsorption buffer for about 1 to about 4 hours at room temperature or 37° C. or overnight at 4° C. by introducing from about 1 μg/ml to about 50 μg/ml (e.g., about 20 μg/ml) of capture antibody on or to the solid phase material. Alternatively, for example, the capture antibody may be immobilized on or to the surface of the solid phase material via an intermediate molecule, such as protein A, A/G, G, or L, adhered on or to the surface of the solid phase material, or the solid phase material may be coated with avidin, streptavidin, etc. to allow binding of a biotinylated capture antibody on or to the solid phase material. Ideally, the amount of capture antibody immobilized on or to the solid phase material should be in excess of the amount of antigen if a quantitative measurement of the antigen is to be made. Once the capture antibody is immobilized on or to the solid phase material, any unbound antibody may be removed by a washing step, and a blocking buffer that contains a blocking agent for binding on or to exposed surfaces of the solid phase material that are not occupied by the capture antibody may be added to avoid non-specific binding of the contents of the test sample or test antigen on or to the solid phase material. Next, a crude or at least partially purified extract sample taken from a food or agricultural source or product to be tested may be added to the solid phase material. For example, the sample may be incubated with the solid phase material for about 1 hour at room temperature or 37° C. If any test antigen, i.e., an antigen from a Pangasius species, is present in the test sample, then it will be bound by the capture antibody and immobilized on or to the solid phase material via the capture antibody. Contents of the test sample that do not bind to the capture antibody may be removed by a wash step. Once any test antigen present in a test sample has been captured by the capture antibody, the remaining steps may be carried out as described above for the direct or indirect immunoassay or ELISA approaches. Finally, the presence of the assayable tag or label on either the primary or secondary detection antibody may be detected by an appropriate assay to indicate whether any test antigen is bound by the capture antibody and immobilized on or to the surface of the solid phase material as a result of the test antigen being present in the test sample derived from the food or agricultural source or product.

According to some embodiments, instead of first immobilizing a capture antibody on or to a solid phase material, the capture antibody may be mixed freely in solution with a test sample that may contain a test antigen. Subsequently, the capture antibody may be immobilized on or to the solid phase material along with any test antigen that may be bound to the capture antibody as a result of being present in the test sample by first coating the solid phase material with a substance that will specifically bind immunoglobulin proteins, such as Protein A, A/G, G, or L. Once the capture antibody and any test antigen bound to the capture antibody are immobilized on or to the solid phase material and washed, the remaining steps may proceed as described elsewhere herein.

According to some embodiments, instead of immobilizing a capture antibody directly on or to a solid phase material, a second capture antibody may be directly immobilized on or to the solid phase material and used to bind and immobilize the capture antibody. Once the capture antibody is indirectly immobilized on or to the solid phase material via the second capture antibody and washed, the remaining steps for the sandwich assay may proceed as described elsewhere herein. This format may sometimes be referred to as a “double sandwich” immunoassay or ELISA.

According to some embodiments, each of the direct, indirect, and/or sandwich immunoassay or ELISA approaches may be further conducted as an inhibition or competition assay. These competition and inhibition approaches may be performed as described above for direct, indirect, or sandwich assays except for the introduction of a competing antigen that will compete with the test antigen for binding. Both competition and inhibition techniques are generally based on observing deviations caused by the addition of a test sample containing a test antigen from levels or amounts of an assayable tag or label that would be detected in the absence of sample or test antigen, such as a control sample or solution that does not contain the test antigen. In other words, if a test antigen is present in a test sample, then the level or amount of an assayable tag or label detected would be lowered as a result of being sequestered and removed by binding of the test antigen to a primary detection antibody or capture antibody—i.e., the level or amount of an assayable tag or label will have an inverse relationship to the amount of the test antigen present in a test sample. Therefore, it may be necessary to separately determine the amounts of assayable tag or label that would be present in the absence of the test sample or test antigen, such as with a control sample or solution that does not contain the test antigen, so that such values may be compared to the amounts of assayable tag or label present when a test sample, which may contain the test antigen, is added. The difference between the inhibition assay approach and the competition assay approach is whether a test sample that may contain a test antigen is (1) first premixed with a primary detection antibody before addition to a solid phase material having a competing antigen immobilized thereto or thereon or a competing antigen bound by an immobilized capture antibody, or (2) added simultaneously with a primary detection antibody on or to a solid phase material having an immobilized competing antigen or a competing antigen bound by an immobilized capture antibody, respectively.

According to some embodiments, a direct or indirect competition assay may be used. A test sample that may contain a test antigen, along with a primary detection antibody, are added to a solid phase material having a competing antigen already immobilized on or to the surface of the solid phase material. The competing antigen may be immobilized on or to the solid phase material in a coating buffer as described above, and a blocking buffer that contains a blocking agent may be added to the solid phase material prior to addition of the sample or primary detection antibody to avoid non-specific binding of the test sample contents or primary detection antibody on or to the solid phase material. Depending on whether a direct or indirect competition assay is used, an assayable tag or label may be attached to, or associated with, either the primary or secondary detection antibody. If free (unbound) test antigen is present in the test sample, then it will bind to the primary detection antibody and thus block the ability of the primary detection antibody to bind the competing antigen immobilized on or to the surface of the solid phase material. During a subsequent washing step, any primary detection antibody that is bound to free (unbound) test antigen present in the sample will be washed away. Thus, the presence of a test antigen in a test sample will reduce the amount or level of an assayable tag or label detected by an appropriate assay compared to a controlled amount or level for the competing antigen measured in the absence of any test sample or test antigen. The controlled amount or level of assayable tag or label may be separately determined in the absence of test sample or test antigen so that such values may be compared to the amounts of assayable tag or label present when a test sample that containing a test antigen is added.

According to some embodiments, a direct or indirect inhibition assay may be used. This approach may be conducted in much the same manner as described for the direct or indirect competition assay, except that a test sample, which may contain a test antigen, is first premixed and incubated with the primary detection antibody prior to their combined addition to the solid phase material having a competing antigen already immobilized on or to its surface, such that any test antigen may bind to the primary detection antibody without competition from a competing antigen. Subsequently, the test sample, which may contain a test antigen, and the primary detection antibody are added to the competing antigen immobilized on or to the surface of the solid phase material. Depending on whether a direct or indirect competition assay is used, an assayable tag or label may be associated with either the primary or secondary detection antibody. If free (unbound) test antigen is present in the test sample, then it will bind to the primary detection antibody and thus block the ability of the primary detection antibody to bind to the competing antigen immobilized on or to the surface of the solid phase material. During a subsequent washing step, any primary detection antibody that is bound to free (unbound) test antigen present in the test sample will be washed away. Thus, the presence of a test antigen in a test sample will reduce the amount or level of an assayable tag or label detected by an appropriate assay compared to a controlled amount or level for the competing antigen measured in the absence of any test sample or test antigen. Again, the controlled amount or level of assayable tag or label may be separately determined in the absence of the test sample or test antigen so that such values may be compared to the amounts of assayable tag or label present when a test sample, which may contain the test antigen, is added.

According to some embodiments, a direct or indirect sandwich competition assay or a direct or indirect sandwich inhibition assay may be used. However, these techniques are much the same as the direct or indirect competition assay or the direct or indirect inhibition assay described above, with the exception that the competing antigen is immobilized on or to a solid phase material by a capture antibody. Considering that using a capture antibody is only another means to immobilize the competing antigen on or to a solid phase material, these techniques are expected to give similar results. The presence of a test antigen in a test sample will reduce the amount or level of an assayable tag or label detected by an appropriate assay compared to a controlled amount or level for the competing antigen measured in the absence of any test sample or test antigen.

According to some embodiments, a modified competition or inhibition assay may be used. Instead of directly labeling an antibody with an assayable tag or label, a competing antigen may be directly linked to the assayable tag or label. This modified assay may be conducted as a sandwich assay with a test antigen and the linked competing antigen competing for binding to a capture antibody that binds to the test antigen. For example, the competing antigen may be a protein or molecule having the same epitope(s) that is present on the test antigen and recognized by the capture antibody. According to a modified inhibition assay, the capture antibody may be immobilized on or to the solid phase material as described above and exposed to a test sample that may contain the test antigen. Any contents of the test sample that did not bind to the capture antibody may be removed by washing. The labeled competing antigen may then be added to the solid phase material to bind any remaining capture antibody not bound by the test antigen. When compared to the controlled level or amount of assayable tag or label measured in the absence of the test antigen, if test antigen is present in the test sample, then the level or amount of assayable tag or label associated with the competing antigen that is bound to the capture antibody will be reduced. The controlled amount or level of assayable tag or label may be separately determined in the absence of sample or test antigen, such as with a control sample or solution that does not contain the test antigen, so that such values may be compared to the amounts of assayable tag or label present when a test sample, which may contain the test antigen, is added. A modified competition assay may be conducted similarly to the modified inhibition assay described immediately above, except that the labeled competing antigen and the test sample may be added to the capture antibody immobilized on or to the solid phase material about at the same time.

For further discussion of theory, principles and practice of using a heterogeneous immunoassay or ELISA to detect an antigen in a sample, see, e.g., Crowther, J. R., “The ELISA Guidebook,” In: Methods in Molecular Biology, 149, (Humana Press, Inc., Totowa, N.J., 2001); Campbell, A. M., “Monoclonal antibody and immunosensor technology,” Laboratory techniques in biochemistry and molecular biology, (van der Vliet, P. C., Ed., Elsevier Science Publishers, 1991); Goslin, J. P. (Ed.), “Immunoassays: A Practical Approach,” (Oxford University Press, 2000); Wild, D. (Ed.) “The Immunoassay Handbook,” (Third Edition, Elsevier, Inc., 2005); and Shepherd et al. (Eds.), “Monoclonal Antibodies: A Practical Approach,” (Oxford University Press, UK, 2000), the entire contents and disclosures of which are hereby incorporated by reference.

### Solid Phase Carrier and Substrate Materials

According to some embodiments of the present invention, a variety of solid phase materials may be used depending on the particular design of the heterogeneous immunoassay or ELISA. For example, the solid phase material may include a polystyrene or polyvinylchloride container or vessel, such as a tube. Alternatively, the solid phase material may include a microtiter plate, such as a 96 well polystyrene or polyvinylchloride microtiter plate. The solid phase material may also include any substrate, support, carrier, etc. such as a plastic or glass material in any shape or dimension, that may be used to immobilize a capture antibody or an antigen of interest either passively, such as by adsorption, or by the use of an intermediate coated on or to the surface of the solid phase material, such as, for example, protein A, G, A/G, or L for a capture antibody, avidin or streptavidin for a biotinylated capture antibody or antigen, etc. The solid phase material may include any plastic, glass or membrane material. For example, a solid phase material may include an immunostick (e.g., dipstick), beads (including magnetic beads), nitrocellulose or nylon membrane, etc. depending upon the particular immunoassay.

To conduct an immunoassay or ELISA according to some embodiments of the present invention, a microtiter plate may be used. For example, a microtiter plate having 96 wells (8×12) may be used. A microtiter plate allows for separate reactions to be carried out in each of the different wells. For example, serial dilutions of reaction components, such as a test sample, antibody, or competing antigen, may be made along a row or column of the microtiter plate to determine the concentration-dependent effects of such components on the amount of assayable tag or label present in the wells. Such microtiter plate dilutions may be used to pretitrate the reaction conditions to optimize conditions for the detection of a test antigen in a test sample. For example, the concentration ranges of capture antibody, primary detection antibody, secondary detection antibody, test sample, and/or competing antigen may be optimized to improve the sensitivity of antigen detection and/or the accuracy for determining test antigen concentrations present in a test sample. In general, concentration ranges of immunoassay components may be optimized for suspected concentrations of the test antigen that might be present in a test sample, such that the suspected concentrations of test antigen in the test sample falls within a linear or dynamic range. By comparing the amount of assayable tag or label present in a well to standard levels of assayable tag or label associated with known amounts of test antigen, the concentration of test antigen in the test sample may be determined. In the case of competition and inhibition immunoassays, it may be necessary to first determine that the amount of assayable tag or label that would exist in a well in the absence of the test sample or test antigen, such as with a control sample or solution that does not contain the test antigen, so that any reduction in the amount of assayable tag or label may be detected and measured when test antigen is introduced by a test sample.

According to some embodiments, immunoassays may also be performed using a variety of other solid phase materials, such as an immunostick, magnetic beads,immunodot assay, etc. each of which may employ the same general concept described above for immunoassays and ELISAs having a direct, indirect, sandwich, competition, etc. format. For example, an immunostick may involve inserting a dipstick made of plastic, glass, etc. into a tube containing a test sample to allow contents of the test sample (1) to passively adsorb on or to the surface of the dipstick or (2) to bind a capture antibody previously immobilized on or to the surface of the dipstick. Subsequently, the dipstick (potentially loaded with a test antigen) may be washed and inserted into a second tube containing a primary detection antibody. If test antigen is immobilized on or to the dipstick, then the primary detection antibody will bind to the test antigen and become immobilized. After washing, the dipstick may then be inserted into another tube allowing visualization of the assayable tag or label, such as by color reaction in a solution containing a reaction substrate. Alternatively, if the primary detection antibody does not contain an assayable tag or label, a secondary detection antibody having an assayable tag or label may also be allowed to bind prior to detecting the presence of the assayable tag or label. The secondary detection antibody may either be contained in the same tube as the primary detection antibody, or the secondary detection antibody may be in a separate tube to allow for a washing step between exposures to the primary and secondary detection antibodies.

According to some embodiments, immunoassays based on the use of plastic, glass, or magnetic beads may also be used. This method may again employ the same general concepts as described above for the various approaches for immunoassays and ELISAs. However, the beads may be suspended in a solution contained in a vessel and concentrated within a region of the vessel either by centrifugation of the beads generally, or by application of an external magnetic field in the case of magnetic beads, to allow the remaining solution to be removed or discarded. These approaches not only aid the washing steps, but may also increase contact of reaction components (e.g., antigen, antibodies, etc.) immobilized on or to the beads with the contents of the various solutions.

According to some embodiments, immunoassays based on the use of an immunodot assay may also be used. These assays are generally based on the immobilization of contents from one or more test samples to separate portions or regions of a membrane, such as a nitrocellulose or nylon membrane, either by passive adsorption or via a capture antibody. With the contents of the one or more test samples immobilized on or to the membrane, the remaining steps may be performed as generally described above for the various approaches for immunoassays or ELISAs. If a test antigen is present in one or more of the test samples, then the presence of an assayable tag or label may be detected using an appropriate assay as a “dot” corresponding to the location on the membrane exposed to such test sample. For example, an insoluble product may be produced and deposited onto locations of the membrane having an assayable tag or label, such as an enzyme.

### Assayable Tags and Labels

An assayable tag or label that may be used according to embodiments of the present invention may include any chemical group known in the art that is capable of later detection, such as by spectroscopic, photochemical, chemical, electrochemical, optical, chromatographic, calorimetric, etc. Such assayable tag or label may include radionuclides or radioactive group, fluorescent dyes or fluorophores, chromogens, chemiluminescers, dyes, enzymes, substrates, cofactors, inhibitors or any other conjugates, such as colloidal gold, colored glass or plastic, biotin, etc. that may either be covalently or chemically linked or bonded to, or otherwise associated with, a primary or secondary detection antibody or competing antigen. The choice of assayable tag or label may depend on practical considerations, such as sensitivity required, ease of conjugation, stability requirements, available instrumentation, disposal provisions, etc.

According to some embodiments, the assayable tag or label may be an enzyme conjugated to either a primary or secondary antibody or a competing antigen. Enzymes may be detected by the conversion of a substrate into a visible or otherwise detectable product. For example, horseradish peroxidase (HRP), alkaline phosphatase (AP or ALP), β-galactosidase, urease, etc. may be used. When horseradish peroxidase (HRP) is used as the assayable tag or label, chromogenic substrates or chromophores (e.g., 3,3′,5,5′-tetramethylbenzadine (TMB); ortho-phenylenediamine (OPD); 2,2′-azino-di-(3-ethylbenzthiazoline-6-sulfonate (ABTS); 5-aminosalicylic acid (5-AS); and diaminobenzidine) or chemiluminescent substrates (e.g., luminal-based products) may be used. Their oxidation by HRP in the presence of hydrogen peroxide results in a color change or chemiluminescence. HRP enzyme generally has a short reaction time and may require the addition of a “stop solution” to halt the reaction and obtain a sensitive and accurate reading.

When alkaline phosphatase (AP) is used as the assayable tag or label, chromogenic substrates or chromophores (e.g., Sigma 104® (Sigma), para-nitrophenyl phosphate (pnpp), or BluePhos® (KPL)) or chemiluminescent (e.g., LuciGLO™ (KPL) or CPSD® with enhancers, such as Emerald-II™ (Tropix)) may be used. Other chromogenic substrates (e.g. phenolphthalein monophosphate, thymophthalein monophosphate, β-glycerophosphate, and uridine phosphate) and fluorigenic substrates (e.g., β-naphthyl phosphate, 4-methylumbelliferyl phosphate, and 3-o-methylfuorescein monophosphate) may also be used. In addition, a combination of 5-bromo-4-chloro-3-indolyl phosphate (BCIP) and p-nitroblue tetrazolium chloride (NBT) may also be used. The AP enzyme tends to have a slower reaction time compared to HRP, but it is not self-limiting and maintains a linear rate of reaction over a longer period of time.

When β-galactosidase is used as the assayable tag or label, the chromogenic substrate or chromophore may include o-nitrophenyl-β-D-galactopyranoside (ONPG), and when urease is used as the assayable tag or label, the chromogenic substrate or chromophore may include bromocresol purple in the presence of urea. Regardless of which enzyme is used, products of chromogenic substrates may either be detected by eye or by measurement of the absorbance or optical density (OD) using a spectrophotometer.

According to some embodiments, the assayable tag or label may be a fluorescent dye or fluorophores conjugated to either a primary or secondary detection antibody or a competing antigen. Any fluorophore that is capable of being conjugated or bound to an antibody or protein may be used with embodiments of the present invention for fluorescent immunoassays. For example, fluorophores may include xanthenes (e.g., rhodol, rhodamine, fluorescein, and derivatives thereof), cyanines, etc. A complete list of examples of fluorophores that may be used with embodiments of the present invention are too numerous to list herein. See, e.g. Hemmila, I. A., “Applications of Fluorescence in Immunoassays,” John Wiley & Sons (1991); and Wood, et al., “Heterogeneous fluoroimmunoassay” In: Principles and Practice of Immunoassay, Stockton Press, NY (1991), the entire contents and disclosure of which are hereby incorporated by reference. Fluorophores generally provide very sensitive detection of an antigen when conjugated directly or indirectly to an antibody that binds to the antigen. However, fluorophores generally require instrumentation to excite the fluorescent label or fluorophore and detect any light emitted therefrom.

According to some embodiments, the assayable tag or label may be a radionuclide conjugated to either a primary or secondary antibody or a competing antigen. Although a variety of radionuclides are available for labeling proteins (e.g., ³H, ³⁵S, ¹⁴C, ³²P), ¹²⁵I is often used since it is easily conjugated and measured. To detect the presence of the radionuclide, any appropriate method may be used, such as by scintillation counting, autoradiography using film, etc.

According to some embodiments, the assayable tag or label may be one or more ligand molecules (e.g., biotin) conjugated to either a primary or secondary detection antibody or a competing antigen for detection using an anti-ligand molecule (e.g., an avidin-like molecule). For example, a biotinylated antibody or protein may be bound with high affinity by avidin, streptavidin, NeutraAvidin (Pierce), etc. (referred to collectively as “avidin-like molecules”), which may themselves be linked to an assayable tag or label. Any of the above identified assayable tags or labels (e.g., enzymes, fluorophores, radionuclides, dyes, etc.) may be conjugated to any of these avidin-like molecules. Because of its small size (only 244 Da), conjugated biotin may be less likely to hinder antibody-antigen interactions. In addition, various conjugated avidin-like molecules are available commercially, which may be used in combination with biotinylated antibodies or competing antigens.

According to some embodiments of the present invention, chromogenic, colorimetric, chemiluminescent, fluorescent, or other visual labels may be detected by microscopy, visual inspection, via photographic film, microtiter plate reader, electronic detectors, etc. such as charge coupled devices (CCDs) or photmultipliers. For example, the IMX™ (Abbott, Irving, Tex.) may be used for a fluorescent immunoassay, and Ciba Corning ACS 180™ (Ciba Corning, Medfield, Mass.) may be used for a chemiluminescent immunoassay. Such instrumentation may be further automated under the control of a computer and associated software. Such computer with associated software may further interpret data gathered using such instruments in addition to determining the amount of chromogenic, calorimetric, chemiluminescent, fluorescent, or other visual label present in an immunoassay solution.

According to some embodiments of the present invention using an indirect immunoassay approach, numerous types of secondary detection antibodies, which may be used to bind a primary detection antibody, are commercially available. However, according to some embodiments using a direct immunoassay approach, it may be necessary to chemically link an assayable tag or label to a newly identified primary detection antibody to allow for detection. Similarly, according to some embodiments relying on a competition or inhibition immunoassay, it may be necessary to chemically link an assayable tag or label to a newly identified competing antigen to allow for its detection. Methods for the conjugation of assayable tags or labels to an antibody or antigenic protein are known in the art and generally rely on the modification of amines, thiols, disulfide linkages, carbohydrates, etc. present on the antibody or antigen. See, e.g., Howard, G. C. et al. (Eds.), “Making and Using Antibodies: A Practical Handbook,” (CRC Press, Boca Raton, Fla., 2007); and Shepherd et al. (Eds.), “Monoclonal Antibodies: A Practical Approach,” (Oxford University Press, UK, 2000), the entire contents and disclosures of which are hereby incorporated by reference.

### Other Immunoassay Techniques

According to some embodiments of the present invention, an immunoprecipitation approach may be used to detect the presence of a test antigen from a Pangasius species, such as tra or basa, in a test sample. This approach is based on the formation of large complexes of molecules nucleated by antibody-antigen interactions that cause such complexes to precipitate out of solution. However, antibody-antigen interactions caused by monoclonal antibodies generally do not have a sufficient number of intermolecular interactions to cause precipitation. Therefore, immunoprecipitation approaches may be limited to polyclonal antibodies and/or antisera having a sufficient number of cross-linking interactions to induce formation of large complexes that precipitate. According to some embodiments of the present invention, any antibody or immunoglobulin-like polypeptides, or functional fragments thereof that are described herein may be used.

Different immunoprecipitation assay formats may also be used including, for example, double immunodiffusion, radial immunodiffusion, and immunoelectrophoresis. These formats are generally based on separate diffusion of (1) one or more test samples that may contain a test antigen or an antigen of interest and (2) at least one sample containing a polyclonal antibody and/or antiserum from distinct positions within a matrix, such as agar, etc. The contents of each test sample are initially in solution, but a precipitate may be formed where samples containing test antigen and antibodies and/or antisera meet by diffusion if polyclonal antibodies and/or antisera are specific for an antigen contained in the one or more samples. The location of any precipitate formed within the matrix may then be used to determine which polyclonal antibody and/or antiserum sample specifically interacts with which test sample, which may be used in turn to determine that such test sample contains the test antigen. Different means may be used to separate different test sample of the contents of a single test sample prior to the diffusion step such as by immunoelectrophoresis or by initially placing different test samples into separate slots or wells present within the matrix. Alternatively, where only a single antigen-containing sample is mixed with a sample containing antiserum or polyclonal antibodies, then the assay may be performed in solution with antibody-antigen interactions detected by the solution becoming turbid or cloudy. Visualization in either case may be improved using a dye.

According to some embodiments of the present invention, another approach closely related to immunoprecipitation, immunoagglutination, may be used to detect the presence of a test antigen from a Pangasius species, such as tra or basa, in a test sample. This approach operates in much the same manner as immunoprecipitation, except that either the antigen or antibody used is presented in a particulate form to aid the formation of insoluble complexes that become visible when antibody and antigen interact and bind. Therefore, this assay may be made more sensitive to complexes having a smaller number of antibody-antigen interactions and cross-linking and may potentially increase the probability that monoclonal antibodies, which may not normally precipitate on their own in solution, to agglutinate if such monoclonal antibodies are conjugated to such particles to facilitate agglutination. For example, the agglutination particle may be a polymer material, a latex particle, a liposome, etc. According to some embodiments of the present invention, any antibody or immunoglobulin-like polypeptides, or functional fragment thereof that are described herein may potentially be used for the immunoagglutination assay. Such immunoagglutination assay may be carried out in solution or by diffusion in a matrix, and visualization may be improved using a dye. In addition, immunoagglutination assays may potentially be conducted in either non-competitive or competitive formats.

According to some embodiments of the present invention, another approach for detecting the presence of a test antigen from a Pangasius species, such as tra or basa, in a test sample may include Western blotting. In general, one or more test samples that may contain an antigen of interest may be separated by polyacrylamide gel electrophoresis (PAGE or SDS-PAGE) and transferred to a membrane, such as nitrocellulose membrane, and probed using antibodies through either direct or indirect means with an assayable tag or label. Western blotting is based on the same general concept for immunoassays described herein, with the membrane acting as the solid phase material. Any test antigen immobilized to the solid phase material (i.e., a membrane) may be detected by either a direct or indirect approach using primary detection antibodies or primary and secondary detection antibodies, respectively. Any antibody or immunoglobulin-like polypeptides, or functional fragment thereof that are described herein may be used as the primary detection antibody to bind an antigen of interest. Methods for carrying out a Western blot are known in the art. See, e.g., Bjerrum, et al., “Handbook of Immunoblotting of Proteins: Experimental and Clinical Applications,” Volume II, (CRC Press, Boca Raton, Fla., 1988), the entire contents and disclosure of which are hereby incorporated by reference. Western blotting approaches may have the advantage of separating antigenic proteins prior to detection to allow for greater distinction between antigens on the basis of size. For example, it is shown herein that monoclonal antibodies T7E10 and T1G11 bind to a different pattern of antigenic bands by Western blot between tra and basa samples. In addition, a two-dimensional gel electrophoresis may be used in combination with a Western blotting approach to improve resolution of antigens or other cross-reacting proteins.

According to some embodiments of the present invention, immunosensors may provide another approach for detecting the presence of a test antigen from a Pangasius species, such as tra or basa, in a test sample. According to some embodiments of the present invention, any antibody or immunoglobulin-like polypeptides, or functional fragment thereof that are described herein may be used with any of the immunosensor assays. In general, these methods are based on a type of signal transduction induced by formation of an antibody-antigen complex. These approaches may use a solid-phase material for immobilization of immunosensor components and may be structured as competitive or sandwich assays. For example, such immunosensors may include (i) electrochemical, (ii) optical, (iii) piezoelectric, or (iv) thermometric approaches. Some of these approaches may have the advantage of allowing detection in the absence of an assayable tag or label. For further description of immunosensors that may be used according to embodiments of the present invention, see, e.g. Ricci, F. et al., “A review on novel developments and applications of immunosensors in food analysis,” Anal Chim Acta 605(2):111-129 (2007); and Luppa, et al., “Immunosensors—principles and applications to clinical chemistry,” Clin Chim Acta 314(1-2):1-26 (2001), the entire contents and disclosures of which are hereby incorporated by reference.

According to some embodiments, electrochemical immunosensors may include potentiometric, amperometric, and conductimetric sensors; however, amperometric sensors are the most common because of their high sensitivity, low cost, and possibility for miniaturization. For example, amperometric sensors may operate by applying a potential between two electrodes to cause oxidation or reduction of an antigen or interest immobilized by an antibody, which may cause a transfer of electrons resulting in a measurable current. Enzymes (e.g., phosphatases, oxidases, peroxidases, etc.) conjugated to either an antibody or competing antigen may also be used with substrates, for example, to cause electrochemical changes in the immediate environment, which may be measured as an electrical current.

According to some embodiments, optical immunosensors to detect the presence of an antigen in a sample may use known methods, including chemiluminescence, light absorbance, fluorescence, phosphorescence, light polarization and rotation, etc. Currently, surface plasmon resonance (SPR), which generally allows sensitive detection without need for an assayable tag or label, is commonly used. Generally speaking, this technique operates as follows: Plane polarized light is directed through a glass prism to a gold/solution dielectric interface over a wide range of incident angles. The intensity of the resulting reflected light is measured against the incident light angle with a detector. At selected incident light wavelengths and angles, the photons of the light waves react with the free electron cloud in the metal film causing a drop in the intensity of the reflected light. The angle at which the drop is maximum (i.e., the minimum of reflectivity) is denoted as the “SPR angle.” This critical angle may be extremely sensitive to the refractive index of the sample in contact with the metal surface—e.g., it is highly influenced by the amount and number of biomolecules immobilized on the surface of the gold metal layer. Adsorption of biomolecules on the metallic film, as well as molecular interactions (e.g., antigen/antibody interactions), induce changes in the refractive index (RI) near the surface, thus giving rise to an angular shift in the resonance angle expressed in terms of resonance units (RU). Basically, according to this form of immunoassay, the surface of the metallic film serves as a solid phase material. This shift is directly proportional to the mass increase and concentration of biomolecules immobilized to the metallic film.

Therefore, a test antigen present in a test sample may be detected and measured according to the SPR technique by determining the angular shift in RUs, and the affinity of the antigen/antibody interaction may also be obtained. According to some embodiments of the present invention, a test antigen present in a test sample may be detected by binding to a capture antibody immobilized on or to the surface of the metal layer or film of the SPR device. Alternatively, the contents of a test sample may be immobilized on or to the surface of the metal layer or film of the SPR device, and the presence of test antigen in the test sample may be detected by binding of a primary detection antibody. However, in contrast to other immunoassay methods described above, binding of unlabeled antigen to an immobilized capture antibody or binding of unlabeled primary antibody to immobilized test antigen may be directly detected without the need for an assayable tag or label conjugated to one of these assay components. However, secondary detection antibodies and conjugates may be used with this approach to enhance detection of a test antigen in a test sample.

Although SPR may be a commonly used optical immunosensor technique, other optical immuosensors may be used including, for example, fluorescent immunosensors based on the use of fiber optics to detect antibody-antigen interactions as a change in optical signal measured through a fiber optic assembly. These approaches rely on the use of an assayable tag or label, such as a fluorophore, conjugated to either an antibody or competing antigen to allow for its detection when localized near one of the fiber optic tips.

According to some embodiments, piezoelectric immunosensors to detect the presence of a test antigen in a test sample may be based on quartz crystal microbalance (QCM). This approach may be carried out in the absence of an assayable tag or label and shares many of the same principles as SPR optical sensors. In general, this approach is based on the measurement of mass changes and physical properties of thin layers deposited on crystal surfaces, such as a quartz crystal, which is a highly precise and stable oscillator. More particularly, when mass is deposited on the surface of a piezoelectric crystal solid phase material, changes in the resonant frequency according to the Sauerbrey equation are detected and may be used, for example, to indicate the binding of an antigen to a capture antibody immobilized to the surface of the solid phase material. Therefore, according to known relationships and formulas, the interaction of an antibody and antigen may be detected according to the QCM immunoassay approach, with either the antibody or antigen immobilized to the surface of the piezoelectric crystal surface and with the surface of the piezoelectric crystal functioning as a solid phase material.

According to some embodiments, a lateral flow test may be used to detect the presence of a test antigen from a Pangasius species, such as tra or basa, in a test sample. Lateral flow tests used for the detection of antibody-antigen interactions may be arranged similarly to heterogeneous immunoassays. According to some embodiments of the present invention, any antibody or immunoglobulin-like polypeptides, or functional fragment thereof that are described herein may be used with a lateral flow test or assay. In general, one end of a strip or length of a solid phase material is inserted into a test sample, and the contents of the sample flow through the solid phase material toward the opposite end of the strip or length of the solid phase material. In general, such solid phase material must be capable of immobilizing a reaction component (e.g., capture antibody, etc.) and allowing a solution to pass through it by capillary action, wicking, or some other chromatographic means. For example, such solid phase material may include paper, nitrocellulose, cellulose acetate, nylon, microporous polymer, etc., or some combination thereof Pore sizes for such solid phase material may range from about 5 microns to about 20 microns. According to some embodiments, the solid phase material may be cast directly onto a polymeric film, or the solid phase material may be laminated directly onto a polymeric film. Such polymeric film may include a polyester film, a Mylar® film (DuPont), or any other similar commercially available film. Prelaminated or precast sheets having such consistency are commercially available (Millipore and Corning).

According to some embodiments of the present invention, a lateral flow assay may require a capture antibody specific for the test antigen to be immobilized on or to the surface of the solid phase material. The immobilized capture antibody may be localized within a line or area of the solid phase material to capture any test antigen that may be present in a test sample as the contents of the test sample flow through the solid phase material. To visualize any test antigen that may be bound to the immobilized capture antigen, a primary detection antibody may also be used. As with other heterogeneous immunoassays, either a direct or indirect approach may be used depending on whether the primary detection antibody is linked to an assayable tag or label or a secondary detection antibody linked to an assayable tag or label, respectively, is used.

Although a variety of different assayable tags or labels may be used with a lateral flow test, assayable tags or labels that are capable of visual detection are often used, such as colloidal gold particles, dyes, colored glass or plastic, etc. According to some embodiments, enzyme conjugates may potentially be used, for example, to convert a chromogenic substrate into a visible or colored product. When an antigen of interest is present in a test sample, it will become bound by the capture antibody immobilized within the line or area of the solid phase material, which may then be detected with one of the detection antibodies. While the immobilized capture antibody may serve as a test line or area for detecting antigen, a second positive control line or area may also be used. For example, the positive control line or area may contain an anti-species antibody specific for the antibody containing the assayable tag or label (i.e., specific for either the primary or secondary detection antibody, respectively, depending upon whether a direct or indirect approach is taken). After exposure to a sample, the positive control line will always display the assayable tag or label regardless of whether test antigen is present in the test sample since the antibody-antibody interaction at the positive control line or area does not rely on the presence of an antigen. For general discussion of lateral flow tests, see, e.g. U.S. Pat. Nos. 7,045,342; 7,344,893; 7,144,742; and 4,943,522, the entire contents and disclosures of which are hereby incorporated by reference.

According to embodiments of the present invention, the presence of a test antigen in a test sample may be detected using an immunohistochemical assay or technique. Immunohistochemical methods for detecting and localizing an antigen in a tissue sample using antibodies are known in the art. In general, this assay involves fixing thin sections of an intact test sample (not extract samples) to allow in situ staining of such fixed section for the presence of antigen. In general, this approach requires thin sections of intact tissue samples to achieve clean punctate staining. Once any antigen is fixed within the tissue sample, it may be detected with one or more detection antibodies by either direct or indirect methods as previously described. According to some embodiments of the present invention, any antibody or immunoglobulin-like polypeptides, or functional fragment thereof that are described herein may be used for an immunohistochemical assay. If a direct approach is used, an assayable tag or label is linked to a primary detection antibody, but if an indirect approach is used, an assayable tag or label is linked to a secondary detection antibody which forms a complex with the antigen via an unlabeled primary detection antibody. For example, a fluorescent tag or label may be used.

According to some embodiments, an immunoarray test may be used to detect the presence of a test antigen from a Pangasius species, such as tra or basa, in a test sample. The immunoarray may be a coated glass slide or silicon wafer containing a high-density array of antibodies, immunoglogulin-like polypeptides, or fragments thereof that are specific to one or more antigens of interest. This approach is particularly suitable when there are two or more antibodies, two or more antigens, and/or two or more arrangements or constructions of antibodies (such as for a sandwich immunoassay) that may be simultaneously analyzed for a particular test sample. Each position within the array may be constructed or arranged as a direct, indirect, sandwich competition, etc. as described herein for heterogeneous immuoassays. For example, one or more antigens of interest may be detected using a fluorescent label to serve as the assayable tag or label. However, any immunosensor approach described herein, such as SPR, may also be used and may be conducted without an assayable tag or label. See, e.g. Twyman, R. M., “Principles of proteomics,” (BIOS Scientific Publishers, New York, 2004), the entire contents and disclosure of which is hereby incorporated by reference.

According to some embodiments of the present invention, other methods known in the art for the detection of antibody-antigen interactions may be used. For example, assays based on mass spectroscopy or dialysis methods may be used to detect the presence of a test antigen from a Pangasius species in a test sample, such as tra or basa, using any antibody or immunoglobulin-like polypeptides, or functional fragment thereof, that is described herein.

### Generation of Antibodies

According to some embodiments of the present invention, immunoglobulin polypeptides and antibodies, such as a capture antibody or a primary detection antibody, that may be used to recognize and bind to an antigen of interest, such as an antigen from a Pangasius species, may include any type of immunoglobulin or immunoglobulin-like polypeptides, or portion(s) or fragment(s) thereof, including, for example, monoclonal, polyclonal, purified, and/or recombinant antibodies as well as antibody fragments and portions of antibodies, as long as the immunoglobulin polypeptide or antibody is capable of binding the antigen of interest with high affinity and specificity.

According to some embodiments, antibodies may be generated, for example, by immunizing an animal with an immunogenic amount of an antigen of interest, such as an antigen from a Pangasius species, which may be emulsified in an adjuvant, such as Freund's complete adjuvant, and/or administered over a period of weeks in intervals that may range from about two to about six weeks. Such methods may include, for example, a first immunization in Freund's complete adjuvant and subsequent immunizations in Freund's incomplete adjuvant (at biweekly to monthly intervals thereafter). Depending on whether monoclonal or polyclonal antibodies are desired, B lymphocyte cells, such as spleen cells, may be extracted from immunized animals and fused with myeloma cells, or serum may be isolated from immunized animals, respectively. Test bleeds may also be taken at regular intervals, such as at fourteen day intervals between the second and third immunizations with production bleeds taken at monthly intervals thereafter.

According to some embodiments, antibodies used to bind, detect, and/or capture an antigen of interest, such as an antigen from a Pangasius species, may include monoclonal antibodies (mAb). Methods for generating monoclonal antibodies are known in the art. In general, antibody-producing B lymphocyte cells collected from an animal that has been injected with an immunogen or antigen may be fused in culture with immortalized myeloma cells having mutations in certain genes that may be used as a basis for selection to form hybridoma cells that continue to produce antibodies. For example, spleen cells may be harvested from an immunized animal and mixed with a myeloma cell line with B lymphocyte and myeloma cells induced to fuse by addition of polyethylene glycol.

For selection purposes, the myeloma cells may contain mutations in the thymidine kinase (TK) and hypoxanthine guanine phosphoribosyl transferase (HGPRT) genes. Normally, animal cells synthesize purine nucleotides and thymidylate de novo from phosphoribosyl pyrophosphate and uridylate, respectively, which are required for DNA synthesis. However, anti-folate drugs, such as aminopterin, may block this de novo pathway, thus forcing the cells to utilize a salvage pathway to synthesize purines and thymidylate from exogenously supplied hypoxanthine and thymidine. Therefore, cells grown in the presence of hypoxanthine, aminopterin, and thymidine (i.e., HAT medium) are able to grow in the presence of aminopterin using the salvage pathway. On the other hand, myeloma cells having mutations in the TK and HGPRT genes are unable to grow under such conditions because of the unavailability of the salvage pathway. Therefore, only mutant myeloma cells that have fused with normal B cells to form hybridoma cells are complemented with normal TK and HGPRT genes allowing them to grow in HAT medium containing aminopterin. B cells that do not become fused with myeloma cells do not survive in culture because they are not immortalized.

Hybridoma technology permits one to explore the entire antibody producing B lymphocyte repertoire of the immune system and to select those cells that produce specific antibodies having the desired binding affinity to a specific antigen of interest, such as an antigen form a Pangasius species. In producing monoclonal antibodies, mutant myeloma cells are fused with a population of B cells extracted from an immunized animal with each B cell, and hence each hybridoma cell, producing a pool of antibodies directed against a single epitope of an antigen. In other words, each fusion event produces a clonal population of hybridoma cells that may be maintained in culture and used to produce a homogeneous pool of antibodies having affinity for a specific epitope. Only those hybridoma cell lines that produce antibodies having a desired affinity for the specific antigen of interest may then be selected or screened from the repertoire of antibody-producing hybridoma cells using any known method in the art (e.g. Western blotting, ELISA, etc.). Thus, monoclonal antibodies provide a pool of identical antibodies directed against a specific epitope of a single antigen. As a result, monoclonal antibodies may have the advantage of improving the quality and accuracy of detection when used as part of an immunoassay, ELISA, or any other assay based on the use of antibodies to detect the presence of an antigen of interest, such as an antigen from a Pangasius species, in a sample by minimizing background effects and non-specific binding to molecules other than the antigen of interest.

According to some embodiments of the present invention, antibodies used to bind, detect, and/or capture a test antigen or an antigen of interest, i.e., an antigen from a Pangasius species, is a monoclonal antibody of the IgG class, such as an immunoglobulin of the IgG1 subclass. According to some embodiments, the capture, primary detection antibody, and/or secondary detection antibody may each be selected from a group consisting of newly identified monoclonal antibodies 7E10.D8.E6 (T7E10), 1G11.D3.E2 (T1G11), 1G11.D3.D12 (F1G11), and 7B8.G11.F1 (F7B8), produced by hybridoma cell lines deposited as ATCC Nos. ______, respectively. The T7E10 and T1G11 monoclonal antibodies are shown herein to bind with specificity and affinity to one or more thermostable antigens present only in extracts of raw or cooked tissue from a Pangasius species, such as tra and/or basa. On the other hand, although the F1G11 and F7B8 monoclonal antibodies are shown herein to bind to one or more antigens in extract samples taken from a Pangasius species, including tra and/or basa, they also cross-react with the same or similar antigens from other species. However, the F1G11 and F7B8 monoclonal antibodies may still be useful. For example, the F1G11 and F7B8 antibodies are shown herein to work well in tandem with the more specific T7E10 and T1G11 antibodies in sandwich immunoassays to detect a thermostable antigen uniquely present in both raw and cooked extract samples taken from a Pangasius species, such as tra and/or basa.

According to some embodiments, antibodies used to bind, detect, and/or capture a specific test antigen or an antigen of interest, such as an antigen from a Pangasius species, may include polyclonal antibodies. Methods for generating polyclonal antibodies are known in the art. Generally speaking, serum is removed from an animal immunized with a specific immunogen or antigen. Such serum will contain antibodies against multiple epitopes and antigens and may further contain antibodies specific for the antigen of interest. Therefore, since a polyclonal antiserum is taken from whole animal blood, such polyclonal antiserum may contain many different antibodies that are capable of binding to a wide diversity of epitopes and antigens, many of which will be unrelated to the antigen of interest. Polyclonal antisera may also contain multiple antibodies that recognize and bind to distinct epitopes of the same antigen (including the antigen of interest) with varying affinity and avidity. To increase their usefulness, such polyclonal antisera may be purified prior to their use by selecting antibodies that bind to the antigen of interest. For example, polyclonal antibodies that recognize and bind to the antigen of interest may be selected by affinity chromatography or purification using at least a portion of the antigen as bait.

According to some embodiments of the present invention, antibodies used to bind, detect, and/or capture a specific antigen of interest, such as an antigen from a Pangasius species, may include functional antibody fragments. Although antibodies of embodiments of the present invention may potentially include IgA, IgD, IgE, and IgM antibody isotypes, and subclasses thereof, IgG antibodies are often used in methods for the detection of antigens.

The basic structure of an IgG antibody is a symmetrical tetrameric Y-shaped complex consisting of two identical light polypeptide chains and two identical heavy polypeptide chains. The heavy chains are linked to one another through at least one disulfide bond, and each light chain is linked to a contiguous heavy chain by a disulfide linkage. Both heavy and light chains may be divided into a series of homologous immunoglobulin (Ig) domains of about 110 amino acids. Each of the Ig domains of the heavy and light chains may be divided into variable (V) or constant (C) regions or domains. In both heavy and light chains, the most N-terminal Ig domain is a variable domain, whereas the remaining Ig domains consist of constant domains. Two antigen-binding sites are formed at the N-terminal ends of each arm of the IgG antibody with the N-terminal ends of each arm comprising the N-terminal variable domains of the heavy and light chains. Within each variable region or domain of the heavy and light chains, there are three hypervariable or complementarity-determining regions (CDRs) that are surrounded by relatively more conserved framework regions. These CDR regions are responsible for much of the amino acid sequence variation between antibodies produced by different cells, which are largely responsible for differences in specificity and affinity to distinct epitopes and antigens.

One method for generating antibody fragments is to subject an antibody to limited proteolytic cleavage or digestion and/or chemical treatment. The proteolytic enzyme, papain, preferentially cleaves the IgG antibody on the N-terminal side of the hinge region to produce three fragments, including two identical Fab (fragment, antigen-binding) fragments and one Fc (fragment, crystalline) fragment. On the other hand, the proteolytic enzyme, pepsin, preferentially cleaves the IgG antibody on the C-terminal side of the hinge region to produce one stable fragment called F(ab′)₂ (two Fab′ fragments held together by an intact hinge region and disulfide bond). The remaining constant regions of the antibody are degraded. Both Fab and F(ab′)₂ fragments retain the antigen-binding variable regions. Fab fragments each have a single antigen-binding site, whereas F(ab′)₂ fragments have two antigen-binding sites. Because Fab and F(ab′)₂ fragments are smaller than intact antibody molecules, more antigen-binding domains may potentially be immobilized per unit area of a solid phase material than when whole antibody molecules are used. According to some embodiments, Fab or F(ab′)₂ fragments of antibodies shown to bind to a test antigen from a Pangasius species, such as tra or basa, may be used as capture antibodies or primary detection antibodies. For example, a capture antibody or primary or secondary detection antibodies may each be selected from Fab or F(ab′)₂ fragments of one of the newly identified monoclonal antibodies 7E10.D8.E6 (T7E10), 1G11.D3.E2 (T1G11), 1G11.D3.D12 (F1G11), or 7B8.G11.F1 (F7B8), produced by hybridoma cell lines deposited as ATCC Nos. ______, respectively.

According to some embodiments of the present invention, antibodies used to bind, detect, and/or capture a specific test antigen or an antigen of interest, such as an antigen from a Pangasius species, may include antibodies or immunoglobulin-like polypeptides produced by any known and available recombinant techniques. In addition, clones encoding antibodies or immunoglobulin-like polypeptides shown to bind the antigen of interest may be further subjected to mutagenesis and selection for antibody evolution in vitro to improve affinity for the antigen of interest. See, e.g., He et al., “Ribosome display: next generation display technologies for production of antibodies in vitro,” Expert Rev Proteomics 2(3): 421-30 (2005); Wark et al., “Latest technologies for the enhancement of antibody maturity,” Adv Drug Deliv Rev 58(5-6): 657-70 (2006); and U.S. patent application Ser. No. 12/026,412, the entire contents and disclosure of which are hereby incorporated by reference. Recombinant methods for engineering and synthesizing antibodies or immunoglobulin-like polypeptides may provide a more stable genetic source compared to hybridoma cell lines and may also be produced more quickly and economically using standard bacterial expression systems.

Once an antibody, such as a monoclonal antibody or an immunoglobulin polypeptide expressed from a library, has been identified as having a desired affinity for an antigen of interest, such antibody may be engineered and designed for expression on the basis of its known sequence. For example, cDNAs may be generated from mRNA isolated from a hybridoma cells that produce an antibody showing specificity and affinity for an antigen of interest, amplified by PCR, and screened for binding to the antigen. Once the cDNA sequence is cloned into a vector, it may be manipulated and engineered as desired by standard recombinant techniques. For example, the sequence may be truncated to include only a functional portion of a full heavy and/or light chain antibody sequence, the antibody sequence may be engineered to have an assayable tag or label, linker sequences may be added to connect functional fragments, chimeric antibodies having portions from different species may be created, etc. The smallest antibody fragment that retains antigen binding is a Fv fragment (variable domain fragment; i.e., a heterodimer of heavy and light chain variable regions). However, Fv fragments are not easily expressed in bacteria and may dissociate without chemical cross-linking.

According to some embodiments, antibodies of the present invention may include an alternative form of a Fv fragment, such as a single chain Fv (scFv) that contains V_{L} and V_{H} domains joined by a linker peptide sequence that are transcribed together as a single transcript to form a single polypeptide chain. Depending on the length and amino acid composition of the linker sequence as well as the number of V_{L} and V_{H} domains joined by linker sequences, then scFv fragments may be either monovalent or multivalent (i.e., referring to the number of antigen-binding sites) between one or more interacting scFv fragments. Because ScFvs are even smaller molecules than Fab or F(ab′)₂ fragments, for example, they may be used to attain even higher densities of antigen binding sites per unit of surface area when immobilized to a solid phase material than possible using whole antibodies, F(ab′)2, or Fab fragments.

According to some embodiments, any functional fragment or engineered version of an antibody that has been shown to bind with specificity to an antigen of interest may be engineered to optimize its binding characteristics and design for expression. For example, Fab and F(ab′)₂ fragments may be generated by recombinant techniques instead of by limited digestion. According to some embodiments of the present invention, any expression system known in the art may be used to express an antibody or immunoglobulin-like polypeptides, or functional fragment thereof, such as in bacteria, yeast, plants, cultured animal cells, etc. See, e.g. Borrebaeck, C., “Antibody engineering,” Breakthroughs in Molecular Biology, (Second Edition, Oxford University Press, Oxford, UK, 1995), the entire contents and disclosure of which are hereby incorporated by reference. For example, when E. coli cells are used for expression, antibodies or immunoglobulin-like polypeptides, or fragments thereof, may be expressed with an appropriate leader or signal peptide sequence, extracted from the periplasmic space, and purified according to standard methods, such as affinity purification using antigen, protein A, protein G, etc. See, e.g. Zola, H., “Monoclonal Antibodies,” The Basics: from background to bench, Kingston, F. (Ed.), (BIOS Scientific Publishers Limited, Oxford, 2000), the entire contents and disclosure of which are hereby incorporated by reference. Alternatively, antibodies or immunoglobulin polypeptides, or fragments thereof, may be expressed and secreted into the surrounding medium using an appropriate vector and bacterial strain. See, e.g., Hoogenboom et al., “Multi-subunit proteins on the surface of filamentous phage: methodologies for displaying antibody (Fab) heavy and light chains,” Nucleic Acids Res. 19(15):4133-37 (1991). However, entry of the antibody or immunoglobulin like polypeptide chains into the oxidizing environment of the bacterial periplasm may be required for proper folding and formation of disulfide bonds.

According to some embodiments, a capture antibody or primary or secondary detection antibody may each be selected from recombinant fragments of heavy and/or light chains of one or more of the newly identified monoclonal antibodies 7E10.D8.E6 (T7E10), 1G11.D3.E2 (T1G11), 1G11.D3.D12 (F1G11), or 7B8.G11.F1 (F7B8), produced by hybridoma cell lines deposited as ATCC Nos. ______, respectively. For example, such capture antibody or primary or secondary detection antibody may each be a scFv synthesized using the variable regions of heavy and/or light chains of the T7E10, T1G11, F1G11, or F7B8 monoclonal antibody encoding sequences.

Methods for preparation antibodies and immunoglobulin-like polypeptides that may be used as part of some embodiments of the present invention are generally known in the art. See, for example, Borrebaeck, C., “Antibody engineering,” Breakthroughs in Molecular Biology, (Second Edition, Oxford University Press, Oxford, UK, 1995); Harlow et al., “Antibodies,” A Laboratory Manual, (Cold Spring Harbor Laboratory Press, N.Y., 1988); Harlow et al., “Using Antibodies,” A Laboratory Manual, (Cold Spring Harbor Laboratory Press, N.Y., 1998); Dennett, R. et al., “Monoclonal Antibodies, Hybridoma: A New Dimension In Biological Analyses,” (Plenum Press, N.Y., 1980); Campbell, A. “Monoclonal Antibody Technology,” Laboratory Techniques In Biochemistry And Molecular Biology, Vol. 13, (Burdon et al. (Eds.), Elsevier, Amsterdam, 1984); Abbas et al., “Cellular and Molecular Immunology,” (W.B. Saunders Co., Philadelphia, Pa., 1997); and Herzenberg et al., “Weir's Handbook of Experimental Immunology,” (5^{th} Edition, Blackwell Scientific Publications, Oxford, 1986), the relevant contents and disclosure of which are hereby incorporated by reference. See also, U.S. Pat. Nos. 4,609,893; 4,713,325; 4,714,681; 4,716,111; 4,716,117; and 4,720,459, the entire contents and disclosures of which are hereby incorporated by reference.

According to some embodiments of the present invention, antibodies used to bind, detect, and/or capture a specific test antigen or antigen of interest, such as an antigen from a Pangasius species, may include antibodies or immunoglobulin-like polypeptides that are identified by screening a library of antibody sequences or a collection of cDNAs. Such screening techniques may include any method for identifying clones that encode antibodies or immunoglobulin-like polypeptides that bind with specificity and affinity to the antigen of interest, including, for example, phage display, ribosomal display, bacterial display, yeast display, etc. or related techniques. See, e.g., Mondon et al., “Human antibody libraries: a race to engineer and explore a larger diversity,” Front Biosci 13: 1117-1129 (2008); Lowman et al., “Selecting High-Affinity Binding Proteins by Monovalent Phage Display,” Biochemistry 30(45):10832-10838 (1991); Clackson et al., “Making antibody fragments using phage display libraries,” Nature 352(6336):624-628 (1991); and Cwirla et al., “Peptides on phage: a vast library of peptides for identifying ligands,” PNAS USA 87(16):6378-6382 (1990), the entire contents and disclosures of which are hereby incorporated by reference.

Phage display methods are generally based on producing genetically altered phage particles, such as recombinant M13 or Fd phages, that display a recombinant protein containing a particular antibody or engineered immunoglobulin-like polypeptides on the surface of the phage particle by fusion with a phage coat protein. Such recombinant phages may be grown and isolated using known methods. By screening individual phage particles for their binding to an antigen of interest, antibodies or engineered immunoglobulin-like polypeptides displayed on the surface of such phage particles may be identified and their sequences readily determined and cloned.

The following provides an exemplary procedure for screening a library of sequences encoding antibodies and immunoglobulin-like polypeptides, and fragments thereof, for binding to an antigen of interest, such as an antigen from a Pangasius species, using a phage display method. For example, candidate sequences may first be made by standard reverse transcriptase protocols to generate cDNA molecules from mRNA isolated from a hybridoma that produces a monoclonal antibody known to bind to an antigen of interest. Such cDNA may then be inserted into a vector and engineered to have desired binding and expression characteristics. cDNA may be inserted into a plasmid by, for example, the method of Maniatis et al., “Molecular Cloning, A Laboratory Manual,” (Second edition, Cold Spring Harbor Laboratory Press, NY, 1989), the entire contents and disclosure of which are hereby incorporated by reference. For example, such cDNA sequence may be engineered into a scFv sequence for expression in vitro.

To make a scFv, the cDNA molecules encoding the variable regions of the heavy and light chains of the monoclonal antibody may be amplified by standard polymerase chain reaction (PCR) methodology using a set of degenerate primers for framework regions of mouse immunoglobulin heavy and light variable regions. Amplified heavy and light chain variable regions may then be linked together with at least one linker oligonucleotide in order to generate a recombinant scFv DNA molecule.

To screen sequences for their ability to bind the antigen of interest, each scFv DNA sequence may be ligated into a filamentous phage plasmid designed to fuse the amplified scFv sequences into the 5′ region of the phage gene encoding a phage coat protein. E. coli cells are than transformed with the recombinant phage plasmids, and filamentous phage grown and harvested. The desired recombinant phages display antigen-binding domains fused to the amino terminal region of the minor coat protein. Such “display phages” may then be passed over immobilized antigen, for example, using the method known as “panning” to adsorb those phage particles containing scFv antibody proteins that are capable of binding antigen. The antigen-binding phage particles may then be amplified by standard phage infection methods, and the amplified recombinant phage population again selected for antigen-binding ability. Such successive rounds of selection for antigen-binding ability, followed by amplification, select for enhanced antigen-binding affinity among the ScFvs displayed on recombinant phages.

Selection for increased antigen-binding ability may be made by adjusting the conditions under which binding takes place to require a higher binding affinity. As mentioned above, enhanced antigen-binding affinity may also be achieved by altering nucleotide sequences of the DNA sequence encoding, for example, the variable antigen-binding domain of the scFv and then subjecting recombinant phage populations to successive rounds of selection for antigen-binding affinity and amplification.

According to some embodiments of the present invention, antibodies and immunoglobulin-like polypeptides, or fragments thereof, may be highly specific for a thermostable antigen from a Pangasius species, such as tra and/or basa. For example, according some embodiments, such antibodies and immunoglobulin-like polypeptides, or fragments thereof, may be used to detect the presence of a test antigen from a Pangasius species, such as tra and/or basa, in a test sample, wherein the sample contains less than about 5% by weight of such antigen. According to some embodiments, such antibodies and immunoglobulin-like polypeptides, or fragments thereof, may be used to detect the presence of a test antigen from a Pangasius species, such as tra and/or basa, in a test sample, wherein the sample contains less than 3% by weight of such antigen. For example, according to some embodiments, such antibodies and immunoglobulin-like polypeptides, or fragments thereof, may be used to detect the presence of a test antigen from a Pangasius species, such as tra and/or basa, in a test sample, wherein the sample contains less than about 1% by weight of such antigen.

### Kits

To facilitate the performance of immunoassays described herein, one or more antibodies identified herein may be provided with additional test reagents and/or items of test equipment in the form a test kit for the rapid, convenient, and reliable detection of a test antigen present in tissue from a Pangasius species, such as tra or basa, in a test sample for regulatory, inspection, scientific, agricultural, etc. purposes. For example, capture antibodies, primary detection antibodies, secondary detection antibodies, substrates, solutions, competing antigens, standards, etc. necessary for the performance of various immunoassays described herein may be conveniently supplied with such test kits. According to some embodiments of the present invention, such kits may include at a minimum a reagent (e.g., a primary detection antibody and/or capture antibody) having specificity and affinity for a test antigen or an antigen of interest, such as an antigen from a Pangasius species, including tra and/or basa. In addition, one or more competing antigens, primary detection antibodies, and/or secondary detection antibodies where appropriate may be further linked or otherwise associated with an assayable tag or label. For example, such kits may include one or more of T7E10, T1G11 F7B8 and/or F1G11 produced by hybridoma cell lines deposited as ATCC Nos. ______, respectively, or a fragment or portion thereof. Such antibodies, competing antigens, standards, etc. may be provided in a solution, as dry powder or precipitate, immobilized on or to a solid phase material, etc.

In addition, such kits may further include a solid phase substrate material, such as a microtiter plate, magnetic or non-magnetic beads, nitrocellulose and/or nylon membrane, plastic or glass materials, such as dipsticks, beads, etc., paper, metal electrode, piezoelectric crystal, etc. in any shape or dimension as appropriate for the particular immunoassay to be performed with such kit. Such solid phase material may be provided, for example, with a capture antibody or competing antigen adhered on or to its surface, such as by adsorption or via an intermediate molecule. Alternatively, for example, such solid phase substrate may be provided with only an intermediate molecule (e.g., protein A, streptavidin, etc.) coated on its surface.

According to some embodiments, such kits may include reagents and/or instructions for performing more than one immunoassay. Such kits may include labeling and/or instructional materials providing directions (i.e., protocols) for the practice of the methods described herein. For example, such instructional materials may describe the detection of thermostable tra and/or basa antigens in a food sample. While the instructional materials typically comprise written or printed materials, they are not limited to such. Any medium capable of storing such instructions and communicating them to an end user is contemplated. Such media may include, for example, electronic storage media (e.g., magnetic discs, tapes, cartridges, chips), optical media (e.g., CD ROM), and the like. Such media may include addresses to internet sites that provide such instructional materials.

According to some embodiments, these kits may comprise nucleic acid constructs (e.g., expression vectors) that encode one or more competing antigens, standards, or engineered antibodies or immunoglogulin-like polypeptides, or fragments thereof, etc. to facilitate their recombinant expression. For example, such kits may include a construct for the expression of a Fab or F(ab′)₂ fragment or a scFv that is specific for an antigen from a Pangasius species, such as tra or basa.

According to some embodiments, such kits may further contain one or more solutions for the performance of an immunoassay. For example, such solutions may include a coating buffer, such as a carbonate buffer, an antibody buffer (for antibody binding to antigen or another antibody), and/or a washing solution, such as phosphate buffer saline (PBS), a reaction solution, such as for the conversion of a substrate a product.

### EXAMPLES

The following non-limiting examples are provided to further illustrate embodiments of the present invention. It should be appreciated by those of skill in the art that the techniques disclosed in the examples that follow represent approaches the inventors have found to function well in the practice of the invention, and thus can be considered to constitute examples of modes for its practice. However, those of skill in the art should, in light of the present disclosure, appreciate that changes may be made to these examples without departing from the spirit and scope of the invention.

### Example 1

### Materials and Methods

### Reagents

Tris-buffered saline, 0.5 M Tris-HCl buffer (pH 6.8), 1.5 M Tris-HCl (pH 8.8), TEMED (N,N,N,N′-tetra-methyl ethylenediamine), Precision Plus Protein Kaleidoscope Standards, 30% acrylamide/bis solution, Tris/glycine buffer, 10× Tris/glycine/SDS buffer, supported nitrocellulose membrane (0.2 μm), and thick blot paper may be obtained from Bio-Rad Laboratories Inc. (Hercules, Calif.). Hydrogen peroxide, horseradish peroxidase conjugated goat antimouse IgG (Fc specific), ABTS (2,2′-azino-bis 3-ethylbenzthiazoline-6-sulfonic acid), and β-mercaptoethanol may be purchased from Sigma-Aldrich Co. (St. Louis, Mo.). Bromophenol blue sodium salt may be purchased from Allied Chemical Corporation (New York). Sodium chloride (NaCl), sodium phosphate dibasic anhydrous (Na₂HPO₄), sodium phosphate monobasic anhydrous (NaH₂—PO₄), bovine serum albumin (BSA), sodium bicarbonate (NaHCO₃), sodium carbonate (Na₂CO₃), citric acid monohydrate, sodium dodecyl sulfate, Tween 20 and all other chemicals, reagents, filters (Whatman No. 1 paper), 96-well polystyrene microplate (Costar 9018) may be purchased from Fisher Scientific (Fair Lawn, N.J.). All solutions may be prepared using distilled deionized pure water (DD water) from a NANOpure DIamond ultrapure water system (Bamstead International, Dubuque, Iowa). All chemicals and reagents are of analytical grade.

### Fish Samples

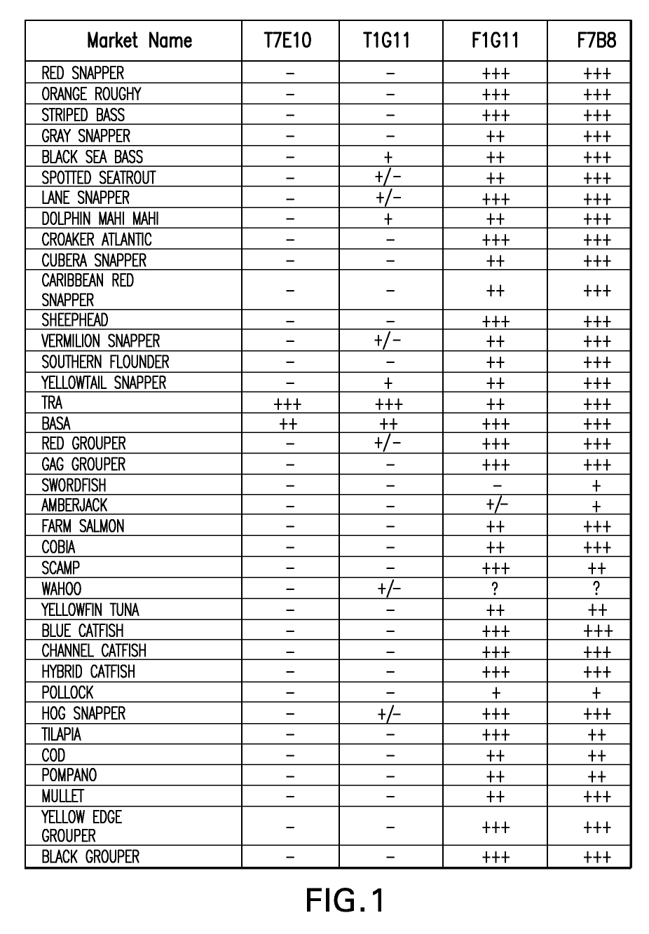

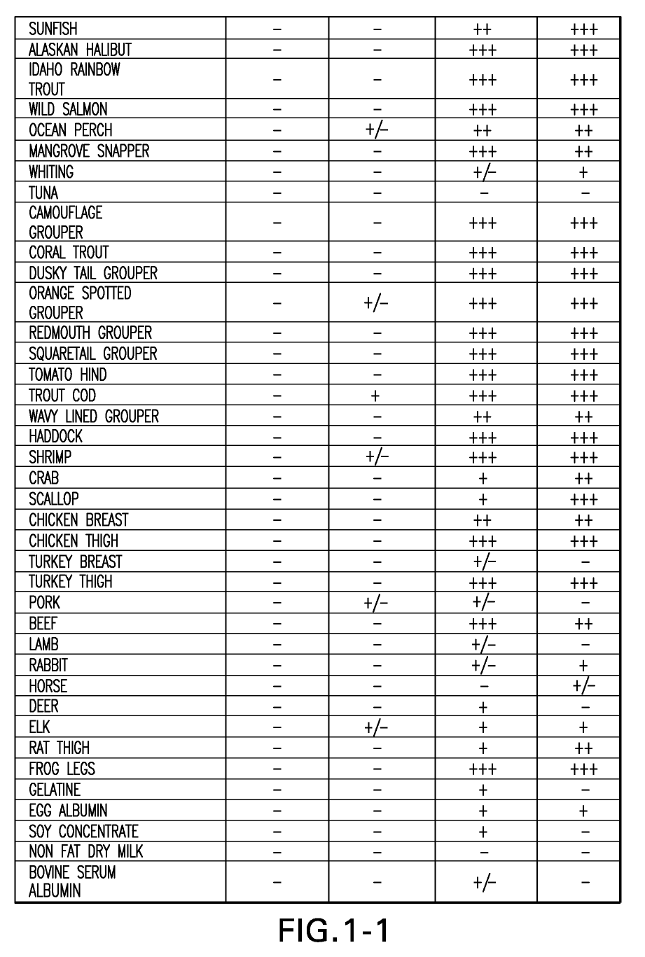

Twenty eight (28) out of fifty five (55) species of common food fish samples tested in these examples were authenticated fish obtained from Florida Department of Agriculture and Consumer Services, Tallahassee, Fla. The remaining fish and shell fish samples that may be either fresh or frozen were purchased from reliable seafood markets in Apalachicola and Tallahassee, Fla. Fresh beef loin, lamb shoulder, pork loin, frozen dressed rabbit, whole turkey and chicken, frozen frog legs were also purchased from local supermarkets. Horse meat was obtained from the School of Veterinary Medicine, Auburn University. All samples were stored at −80° C. until use. The common names for fish and other meat sample sources are provided in the table of FIG. 1.

Protein Extraction from Fish and Meat Samples.

To prepare an extract from a cooked sample, half-frozen fish fillets or meat samples are cut into smaller pieces of desired weight. About 10 grams of the fish or other meat sample from each source are weighed into beakers. The beakers are covered with aluminum foil, sealed with adhesive tape, and heated in a boiling water bath for about 15 min. After cooling at room temperature, the cooked samples are mashed into fine particles using a glass rod. About a threefold saline solution (3:1 ml/g of 0.15 M NaCl) may be used as an extraction buffer (about 0.15 M NaCl) added to the mashed samples (5 ml/g), and the mixture is homogenized for about 1 min at 11,000 rpm. The homogenized samples are then allowed to stand at 4° C. for about 2 hours and centrifuged at 5,000×g for about 30 minutes at 4° C. The supernatants are filtered through Whatman No. 1 filter paper and stored at −20° C. until used. Protein extracts from raw fish samples are prepared by adding about 35 mL of 0.15 M NaCl to about 5 g of minced raw fish in a beaker, and the mixture is homogenized for about 2 min at 11,000 rpm followed by standing at 4° C. for 2 hours. The mixture is then centrifuged and filtered as described above and stored at −20° C. The non-fish samples including poultry and meat were previously grinded when received and prepared in the same fashion as described above for the fish samples. Protein extracts from five non-flesh proteins including gelatin, soy protein concentrate, egg albumin, nonfat dry milk and BSA are prepared by mixing about 1 gram of each in about 10 mL of saline in a beaker. Gelatin and egg albumin were pre-heated in water bath to increase their solubility. These mixtures were then centrifuged and filtered as described above.

### Immunogen Preparation.

A crude protein extract of cooked tra prepared as described above was dialyzed in about 10 mM PBS for about 24 hours using a dialyzing tube with molecular weight cut-off of about 10 kDa. The dialyzed protein extract was filtered through 0.45 μm disposable filter into sterile tubes (about 1 mL/tube). This crude protein extract was used to immunize animals for monoclonal antibody development. This partially purified crude protein extract allows any of the individual thermostable sarcoplasmic proteins in the extract to serve as potential antigens capable of eliciting antibody production. The heat treatment ensures that proteins that remain soluble in the solution after heating are heat-stable because most sarcoplasmic fish proteins become denatured and insoluble after cooking. Removal of small molecules below 10 kDa by dialysis of the protein extract is performed to eliminate impurities and small molecules so that retained thermal-stable proteins in the solution are all immunogenic.

### Immunization.

Using a partially purified crude protein extract from cooked tra as an immunogen, MAbs were developed from hybridomas created from spleen cells isolated from immunized mice and screened for their ability to react and bind to antigens present in tissue samples derived from a Pangasius species, such as tra and/or basa. By using a heated or cooked extract to immunize animals, MAbs may be generated against sarcoplasmic antigenic proteins that are thermo-stable, which may be important for detecting antigens from a Pangasius species, such as tra and/or basa, in a cooked food sample. Indeed, most proteins become insoluble upon heating and were removed from the crude extract used for immunization. Three female BALB/c mice were immunized subcutaneously with about 100 μg/mouse of the dialyzed extract (immunogen) in phosphate buffered saline (PBS) emulsified with an equal volume of Freund's complete adjuvant. Three boost injections prepared in the same manner using Freund's incomplete adjuvant were applied to each mouse at 4-week intervals. Test sera from mice were collected 8 days after each boosting by tail bleeding. The titer of the sera was determined by an indirect ELISA. The mouse showing the highest titer was injected intraperitoneally with 100 μg of the immunogen about 4 days prior to isolation of spleen cells for hybridoma fusion.

### Hybridoma Procedures.

Spleen cells from immunized mice were fused with murine myeloma cells (P3x63.Ag8.653, ATCC CRL 1580) for hybridoma production. The general procedures are known in the art and were followed with some modifications. See, e.g. Kohler et al., “Continuous Cultures of Fused Cells Secreting Antibody of Predefined Specificity,” Nature 256(5517):495-497 (1975). Those hybridomas secreting monoclonal antibodies (MAbs) that react with the target immunogen were selected, cloned twice by limiting dilution, and expanded. An advantage of monoclonal antibody (MAb) development is that impure antigen (e.g., crude extract) may be used for animal immunization due to the ability to select hybridoma cell clones secreting MAbs that specifically react and bind to an antigen of interest. Western blotting may then be used to characterize and test identified MAbs to confirm which antibodies react specifically with thermo-stable antigens present in both raw and cooked tissue samples from a Pangasius species, such as tra and/or basa.

Although MAbs showing positive reaction to antigens include both IgM and IgG classes, only IgG class of MAbs are selected because IgM antibodies are generally more difficult to purify and store. IgG selection may be achieved using IgG γ-chain specific secondary antibody as a probe in the ELISA screening procedures. The subclass of the MAbs may also be determined with a commercial kit mouse MAb isotyping kit (Sigma) according to the manufacturer's instructions. MAb IgGs were purified using a Bio-Rad Protein A Cartridge with the Bio-Rad Econo LC system. The concentration of IgG in the final preparation was determined by UV absorption at 280 nm. The purified MAbs were titrated against the antigen by indirect ELISA to confirm immunoreactivity, and the sizes of antigenic targets for each MAb were determined by Western blotting.

The initial screening of MAbs against the immunogen (i.e., cooked or thermo-stable tra proteins) was performed using indirect ELISA. For secondary selection, the positive cells from the initial screening were expanded and the supernatants screened for reactivity with the native (i.e., raw or uncooked) tra protein extract to ensure the MAbs reacted to both native and heated antigenic proteins. Only hybridomas having positive reaction to only tra and/or basa antigens were selected. Four monoclonal antibodies, 7E10.D8.E6 (T7E10), 1G11.D3.D12 (F1G11), 1G11.D3.E2 (T1G11) and 7B8.G11.F1 (F7B8) were selected and cloned. All of these four MAbs belong to subclass IgG1. Two of the MAbs (T7E10 and T1G11) are shown to react specifically with antigens from a Pangasius species, such as tra or basa, and two other MAbs (F7B8 and F1G11) are shown to cross-react with antigens from other species. However, as provided below, the cross-reactive MAbs may be useful in sandwich assays for tra or basa antigens in combination with more specific MAbs.

### Indirect ELISA.

About 100 μL of diluted sample protein extract containing 2 μg of soluble protein in 0.06 M carbonate buffer (pH 9.6) was coated onto the wells of a 96-well polystyrene microplate (Costar 9018, Fisher) and incubated at 37° C. for about 2 h. The plate was then washed three times with PBST [0.05% v/v Tween-20 in 10 mM PBS, pH 7.2] and incubated with 200 μL/well of blocking solution (3% NFDM in PBS) at 37° C. for about 2 h, followed by another washing step. About 100 μL undiluted or appropriately diluted MAb supernatants in antibody buffer (1% w/v BSA in PBST) were added to each well and incubated at 37° C. for about 2 h. After washing three times with PBST, diluted (1:3000 in antibody buffer) horseradish peroxidase-conjugated goat anti-mouse IgG-Fc specific solution was added. The plate was incubated at 37° C. for about 2 h and washed five times before addition of the substrate solution (22 mg of ABTS and 15 μL of 30% hydrogen peroxide in 100 mL of 0.1 M phosphate-citrate buffer, pH 4.0). The color was developed at 37° C. for approximately about 20 min. The enzyme reaction was stopped by adding 0.2 M citric acid solution, and the absorbance was measured at 410 nm using a microplate reader (Model MQX200R, BioTek). This optimized indirect ELISA procedure was used for hybridoma screening as well as MAb characterization.

### SDS-PAGE and Western Blot.

Sodium dodecyl sulfate—polyacrylamide gel electrophoresis (SDS-PAGE) is performed according to known methods to separate the soluble proteins in different sample extracts based on their molecular weights. Briefly, soluble proteins (3 μg of protein in 10 μL per lane) from each samples may be loaded on the polyacrylamide stacking gel (5%, pH 6.8) and separated on the polyacrylamide separating gel (14%, pH 8.8). The gel may then be subjected to electrophoresis at 200 V for about 45 min using a Mini-Protein 3 electrophoresis cell (Bio-Rad, 161-3301) connected to a power supply (Model 3000, Bio-Rad). The Western Blot analysis may then be carried out according to known methods. After separation of proteins on the polyacrylamide gel by SDS-PAGE, protein bands may be transferred electrophoretically (1 h at 100 V) from the gel to nitrocellulose membranes using a MiniTrans-Blot unit (Bio-Rad). Upon completion of the transfer, the membrane may then be washed with TBST (20 mM Tris, 500 mM NaCl, 0.05% Tween-20, pH 7.5), blocked with 1% BSA in TBS, and incubated with a MAb supernatant diluted 1:1 in antibody buffer for about 2 hours at room temperature. The excess antibody reagent may be removed by washing with TBST, and the membrane may be incubated with goat antimouse IgG-alkaline phosphatase conjugate diluted 1:3000 in antibody buffer for about 1 hour at room temperature. After washing, the membrane may be incubated with 5-bromo-4-chloro-3-indolyl phosphate/p-nitroblue tetrazolium chloride (BCIP/NBT) in 0.1 M Tris buffer at pH 9.5 to develop the color. The color reaction may be stopped by washing the membrane with distilled water. The appearance of staining indicates bands that bind specifically to the MAb. Prestained broad range protein standards (Precision Plus Protein Kaleidoscope Standards, Bio-Rad, 161-0375) are also used as molecular weight markers.

### Example 2

### Characterization of Monoclonal Antibodies

### Species Specificity and Cross Reactivity

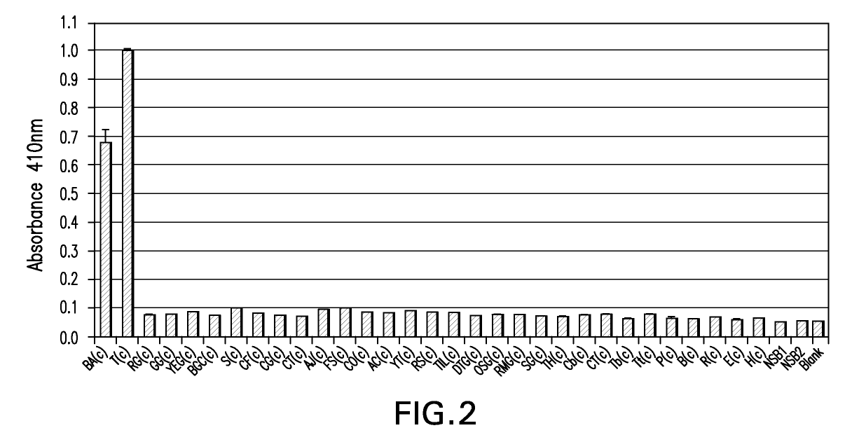

Species specificity of the newly produced MAbs were determined by testing each MAb supernatant with raw and cooked protein extracts from 58 common fish and shelfish species, 13 meat extracts, and 5 other protein extracts (bovine serum albumin, egg albumin, gelatin, and non-fat dry milk proteins) using indirect ELISA procedures. Four IgG class MAbs showing strong positive immunoreactivity to both raw and cooked tissue extracts from Pangasius fish, such as tra and/or basa, were selected. Among these four MAbs, T7E10 binds specifically to only basa and tra antigens without any cross-reactivity with other seafood, meat or food proteins tested (see FIG. 2). Similarly, T1G11 binds specifically to basa and tra antigens with weak cross-reactivity with only a few other fish species. Both of these MAbs showing specificity for Pangasius antigens also show stronger reactivity with tra antigens compared to basa antigens. The two other MAbs, F7B8 and F1G11, were cross-reactive to antigens from all fish and shellfish species as well as some mammalian species. FIG. 1 provides a summary of species-specificity and cross-reactivity of all four MAbs identified against protein extract samples derived from cooked (100° C. for 15 min) fish, shellfish, meat and other protein sources by indirect ELISA. Because cooked samples are enriched with thermo-stable proteins, the indirect ELISA test shows a much weaker signal for antigens from raw samples compared to cooked samples as a fraction of total protein (data not shown). To improve signal with raw food samples using indirect ELISA, raw sample extracts may be heated for a few minutes to remove heat-labile proteins before coating the extract on a solid phase substrate, such as a microtiter plate.

### Thermostable Antigenic Components.

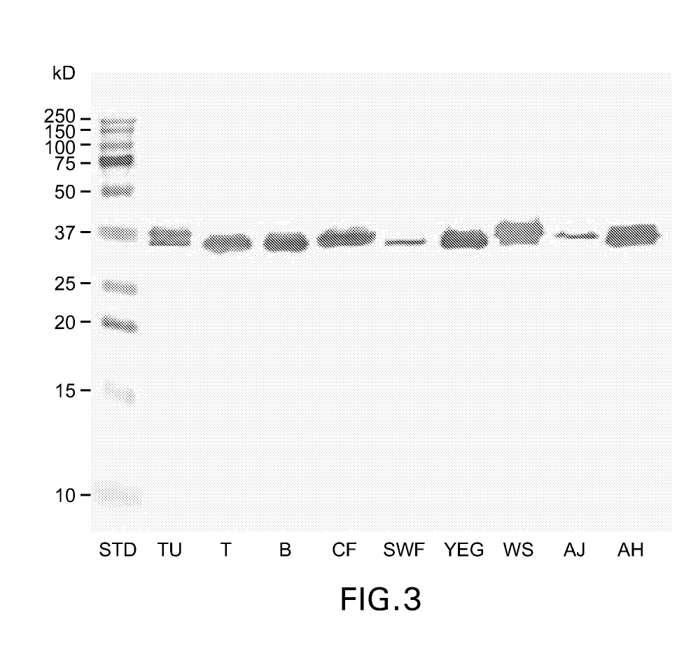

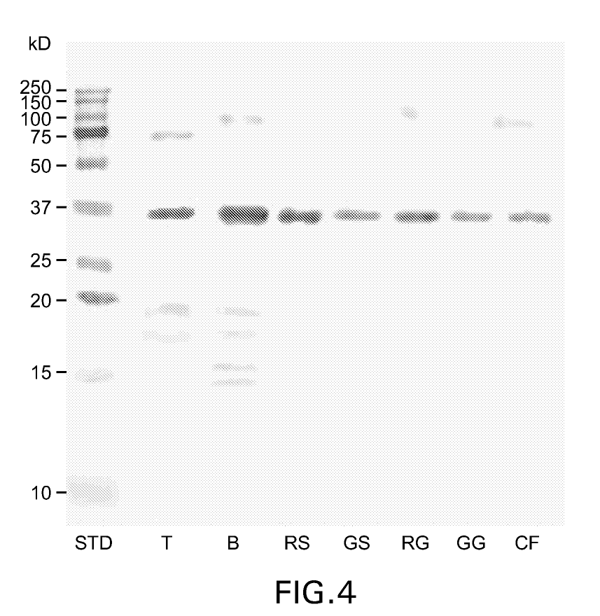

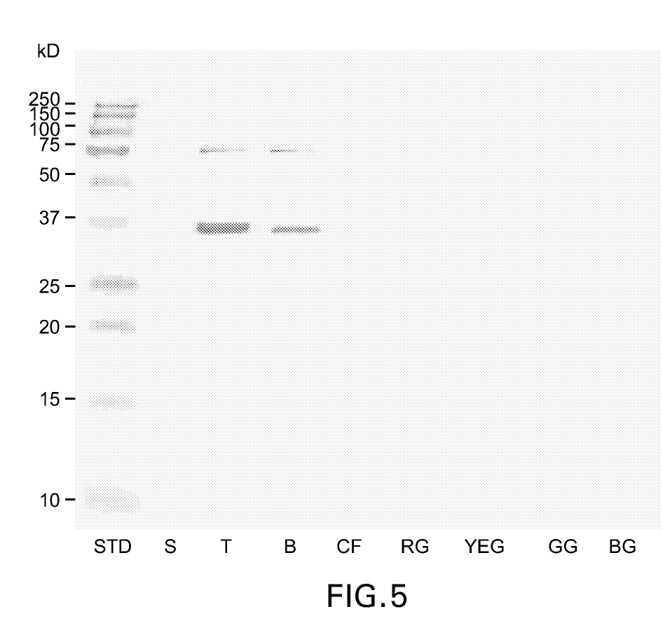

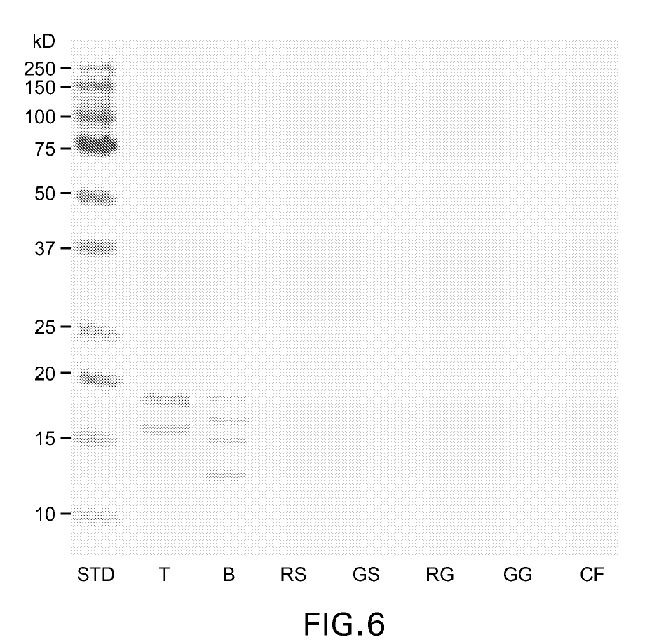

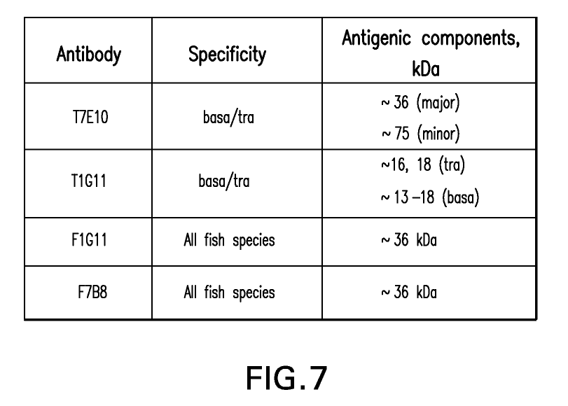

Western blotting was performed to determine the sizes of antigenic proteins in tissue extracts from different fish samples, including tra and basa. Both F7B8 and F1G11 MAbs are shown to react with a major band around 36 kDa in extracts from all fish species tested. See FIGS. 3 and 4 In contrast, the two Pangasius-specific MAbs, T7E10 and T1G11, exhibited specificity for antigens present only in tissue extracts from a Pangasius species, such as tra or basa. Although the two Pangasius-specific MAbs have similar specificity for tra and basa antigens as shown by ELISA, the patterns and sizes of bands detected by Western blot using the two Pangasius-specific MAbs are different. For example, MAb T7E10 binds two major proteins of about 36 and 75 kDa in cooked tra and/or basa extracts (see FIG. 5), while MAb T1G11 recognizes several smaller proteins between about 13 and 18 kDa in the basa and tra extracts (see FIG. 6). No antigenic protein bands were detected with either T7E10 or T1G11 in any of the other non-Pangasius samples tested by Western blot. See FIG. 7 for a summary of sizes of antigenic proteins specifically recognized and bound by each MAb. Without being bound by any theory, it is believed that T7E10 only recognizes the 36 kDa antigen in tra or basa samples because it is directed to a non-conserved epitope on the antigen. In contrast, the F1G11 and F7B8 antibodies are believed to bind the 36 kDa antigen in all species because it recognizes a conserved epitope on the antigen.

These results showing the immunoreactivity of each MAb by Western blot generally agree with data obtained from indirect ELISA. Because the T1G11 MAb recognizes a different pattern of antigenic peptide in tra and basa fish extracts, the T1G11 MAb may further be used to distinguish between basa and tra fish antigens present in a test sample. These data show the potential for using one or more MAbs identified herein to construct immunoassays for the rapid and reliable identification of raw or cooked tissue from a Pangasius species, such as tra and/or basa, in a food sample by specific recognition of Pangasius-specific antigens. In addition, these MAbs may also be valuable for the study of chemical, biological, and physiological properties of these species-specific and thermo-stable sarcoplasmic protein antigens identified herein.

Without being bound by any theory, preliminary data indicates that the approximately 36 kDa protein antigen detected by the T7E10 antibody in tra and basa samples and by the F1G11 and F7B8 antibodies in all fish species is the protein tropomyosin. This is based on a few observations: (i) tropomyosin is known to be thermostable; (ii) tropomyosin is known to be about the same size; and (iii) the cross-reactive antibodies bind to a commercially purified chicken tropomyosin protein. In addition, the approximately 75 kDa band detected by the T7E10 antibody in tra and basa samples is believed to be a dimer of tropomyosin that escaped the dissociation effect imposed by the SDS reagent in the procedure. In addition, without being bound by any theory, the approximately 16 and 18 kDa protein antigens detected in tra samples and the 13-18 kDa protein antigens detected in basa samples by the T1G11 antibody are believed to include the protein parvalbumin based on its known size and thermostability.

### Example 3

### Development of Sandwich ELISA for Tra and Basa Antigens

### Epitope Comparison

To select a suitable pair of MAbs for a sandwich ELISA, it may be necessary to use two MAbs which bind to a common antigen but on the different sites (epitopes) that will not inhibit each other's binding properties. Because three of the four MAbs identified herein bind to a common 36 kDa protein, these antibodies may potentially be used together in an ELISA test. However, epitope mapping for all four MAbs was carried out by performing the additivity test as previously described with some modifications. See, e.g. Friguet et al., “A convenient enzyme-linked immunosorbent assay for testing whether monoclonal antibodies recognize the same antigenic site. Application to hybridomas specific for the beta 2-subunit of Escherichia coli tryptophan synthase” J. Immunol. Methods 60:351-358 (1983), the entire content and disclosure of which is hereby incorporated by reference.

For the additivity test, microplate wells may be coated with about 100 μl of cooked fish extract containing 0.5 μg of soluble proteins diluted in a carbonate buffer (pH 9.6). The plate may be incubated for about 2 hours at 37° C. followed by three washings with PBS containing 0.05% Tween-20 (PBST). After washing, each well of the microplate may be blocked with about 200 μl of blocking buffer (1% BSA in PBS) and incubated at 37° C. for about 2 hours. Three reaction solutions of primary antibody are separately prepared for each pair-wise combination of MAbs: (i) supernatant of MAb#1 diluted 1:3 in antibody buffer (1% w/v BSA in PBST); (ii) supernatant of MAb#2 diluted 1:3 in antibody buffer; and (iii) both MAb#1 and MAb#2 diluted together in a 1:1 ratio in antibody buffer. After washing with PBST, about 100 μl of each of the three MAb reaction solutions for each pair-wise combination of MAbs may be added to separate wells and allowed to incubate at 37° C. for about 2 hours. After another washing step, 100 μl of a secondary detection antibody (e.g. horseradish peroxidase-conjugated goat anti-mouse IgG-Fc specific diluted 1:3000 in antibody buffer) may be added to each well of the plate and incubated at 37° C. for about 2 hours. After additional washes, substrate solution (e.g., 22 mg of ABTS and 15 μL of 30% hydrogen peroxide in 100 ml of 0.1 M phosphate-citrate buffer, pH 4.0) may be added for color development at room temperature for about 25 min. The enzyme reaction may then be stopped by adding 0.2 M citric acid solution, and the absorbance at 410 nm read for each well using microplate reader (Model MQX200R, BioTek).

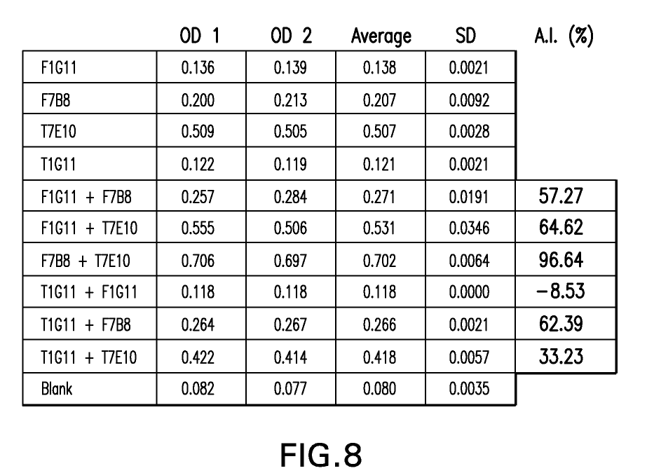

Epitope comparison was performed on all four MAbs to determine which two MAbs may have complimentary epitopes suitable for use together in a sandwich ELISA test. When two MAbs are tested together in a well, their binding sites may or may not be overlapping on a common antigen molecule. The following equation may be used to calculate the Additivity Index (A.I.) for any combination of two MAbs tested:

$${A}_{x} {I}_{x} = \frac{{A}_{1 + 2} - \frac{{A}_{1} + {A}_{2}}{2}}{{A}_{1} + {A}_{2} - \frac{{A}_{1} + {A}_{2}}{2}} \times 100 = ( \frac{2 {A}_{1 + 2}}{{A}_{1} + {A}_{2}} - 1 ) \times 100$$

wherein A₁, A₂ and A₁₊₂ are the absorbance readings reached, in the additivity test, with MAb#1 alone, MAb#2 alone, or both MAb#1 and MAb#2 combined together. The absorbance readings for each of the four MAbs alone as well as absorbance readings for pair-wise combinations of two MAbs are measured, and A.I. (%) values for each MAb combination are calculated. See FIG. 8. If two MAbs bind to the same epitope on a common antigenic protein (i.e., they perfectly compete for binding to the antigen), then the A₁₊₂ should be equal to the mean value of A₁ and A₂, and the A.I. value will be about 0%. However, if the pair of MAbs bind to different non-overlapping epitopes on the antigenic protein, then the A₁₊₂ should be the sum of A₁ and A₂, and the A.I. will be about 100%. Generally, two antibodies are considered to either share the same binding side or have overlapping binding sides to some degree if they have an A.I. value that is below about 50%. On the other hand, if the A.I. is above 50%, then the two antibodies are not considered to have epitopes that significantly overlap to inhibit binding by the other antibody.

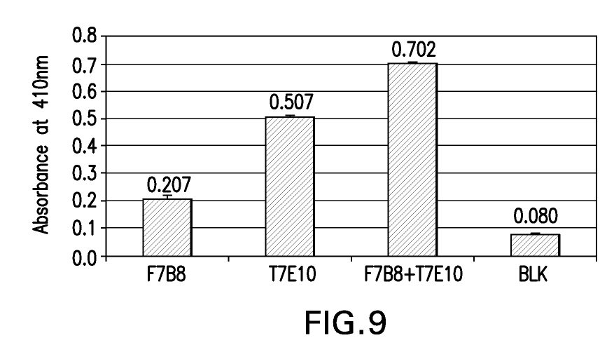

Experiments were performed in duplicate for each antibody alone and each combination of antibodies to obtain two optical density (O.D.) readings, and from these values, an average O.D. reading with standard deviation (S.D.) was obtained. See FIG. 8. Among the four antibodies tested, it is shown that binding of T7E10 to antigen does not inhibit binding of F7B8 because their shared A.I. value is about 96.64% (i.e., nearly 100%). This combination shows the highest A.I. value among the different MAb combinations. See FIGS. 8 and 9. Therefore, these two antibodies are suitable for use together in a sandwich ELISA because they recognize the same thermostable antigenic protein (˜36 kDa) in both raw and heat-treated Pangasius fish extracts (as revealed by Western blot analysis), but their binding is complementary as determined by the Additivity Test.

### Sandwich ELISA.

Optimization and development of an ELISA test based on this antibody combination (F7B8+T7E10) was performed to determine optimal dilutions for each antibody, incubation periods, and choice of blocking buffer. The following procedure provides an example of a sandwich ELISA using optimized conditions with the T7E10 MAb biotinylated to improve detection. According to this sandwich ELISA approach, F7B8 is used as the capture antibody, and T7E10 is used as the primary detection antibody. About 100 μL of capture antibody (purified F7B8) may be diluted in PBS to 0.5 μg protein per 100 μL per well, may be coated on the wells of the microplate and incubated at 37° C. for about 2 hours. The plate may then be washed about three times with PBST and incubated for about 1 hour at 37° C. with about 200 μL of the blocking buffer. After washing twice with PBST, about 100 μL of control samples and undiluted protein samples may be added to the plate and incubated for about 2 hours at 37° C. The plate may then be washed and incubated for about 2 hours at 37° C. with 100 μL of the primary detection antibody (biotinylated MAb T7E10) diluted 1:1000 in antibody buffer (corresponding to 0.05 μg protein per 100 μL). The plate may again be washed about three times and incubated for about 1 hour at 37° C. with 100 μL of streptavidin-conjugated peroxidase enzyme diluted 1:1000 in antibody buffer. After another washing step, the plate may then be incubated with about 100 μL of ABTS enzyme substrate for about 20 min at 37° C. for development of the color reaction. The enzyme reaction may then be stopped by addition of 100 μL of 0.2 M citric acid, and the absorbance may be read at 410 nm.

### Selection of Extraction Solution

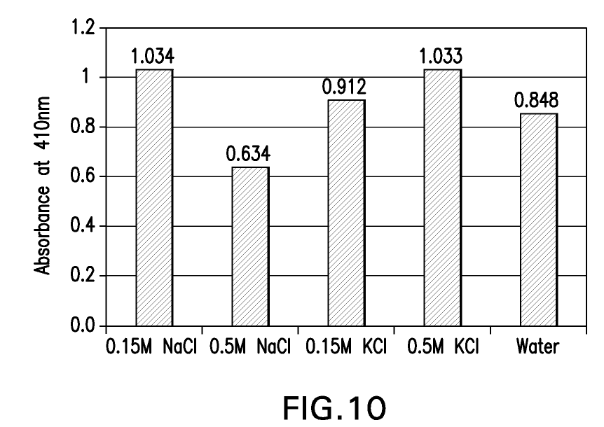

To further optimize the sandwich ELISA procedure, different extraction solutions were tested to determine which is most effective for the sandwich ELISA. Five commonly used solutions including water, 0.15 M NaCl, 0.5 M NaCl, 0.15 M KCl, and 0.5M KCl were tested for extraction using the optimized sandwich ELISA. Among these five, 0.15 M NaCl produces the strongest ELISA signals, followed by 0.5 M KCl, 0.15 M KCl, water and 0.5M NaCl in descending order indicating extraction by 0.15 M NaCl may yield the highest amount of the target protein from a sample. See FIG. 10. These data also indicate that the antigenic protein is abundant in the sarcoplasm portion of muscle cells since higher concentrations of salt solution are used to extract myofibril proteins.

### Specificity of the Sandwich ELISA

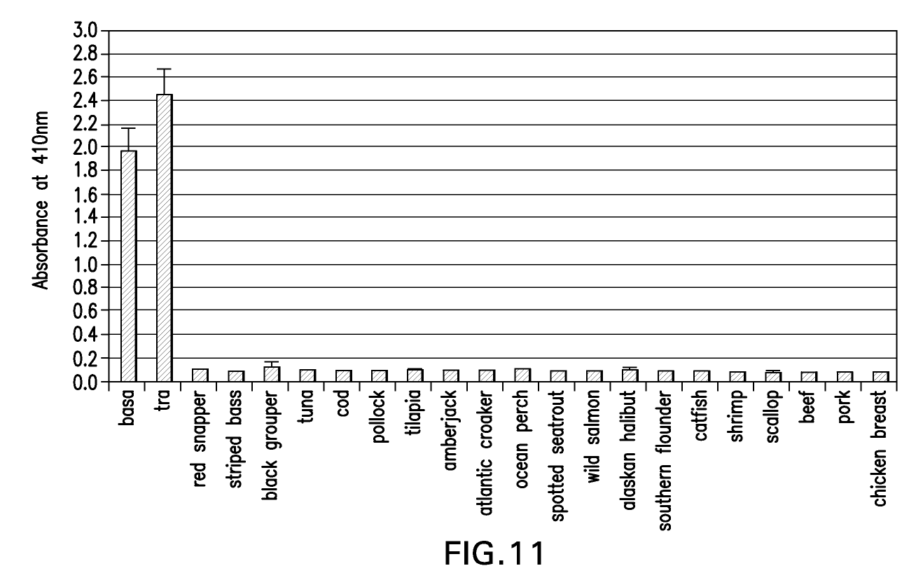

After selection of MAbs F7B8 and T7E10 for the sandwich ELISA and optimization of reaction conditions, it was further determined that using the F7B8 antibody as the capture antibody and a biotinylated T7E10 antibody as the primary detection antibody gave a stronger reaction to basa and tra in a sample and clear negative reaction with all other non-Pangasius tissue samples than the reversed construction model. Using this molecular construction or arrangement of F7B8 and T7E10 antibodies for the sandwich ELISA test under optimized conditions, tissue extracts from tra, basa, and other non-Pangasius species were tested to determine the specificity and strength of the ELISA test. These data clearly show high strength and specificity for only tissue extracts obtained from cooked tra or basa fish samples among all samples tested, including a total of 55 fish and shellfish species, 14 non-fish animal sources, and 5 other food protein sources, without any slight cross reactivity with other fish or non-fish protein samples. See FIG. 11.

Due to its strength and clarity, this sandwich ELISA technique using newly developed MAbs specific for antigens from a Pangasius species, such as a tra or basa, may be used to reliably detect the presence of significant amounts of tra and/or basa fish in a sample. Unlike other analytical methods, no authenticating fish standards need to be tested alongside unknown samples for comparison because of the species specific nature of the assay. Furthermore, the relatively easy sample preparation needed in conjunction with its capability for high throughput analysis, enable this immunoassay to be used for routine analysis of large numbers of samples and for the development of rapid test kits, such as lateral flow assays, immunosticks, etc. that may be amenable to on-site field use. Indeed, such assay would provide a powerful tool to discourage the illegal practice in the market and to enforce the labeling regulations for consumer protection.

All documents, patents, journal articles and other materials cited in the present application are hereby incorporated by reference in their entirety. Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart therefrom.

1. A hybridoma cell line deposited as one of ATCC Accession Nos. PTA-9722, PTA-9723, PTA-9724, and PTA-9725.

2. An antibody, comprising T7E10, T1G11, F7B8, or F1G11 produced by one of the hybridoma cell lines of claim 1, or a fragment or portion thereof.

3. The antibody of claim 2, further comprising an assayable tag or label.

4. The antibody of claim 3, wherein the antibody is chemically bonded to the assayable tag or label.

5. The antibody of claim 2, wherein the antibody comprises T7E10, produced by a hybridoma cell line deposited as ATCC Accession No. PTA-9722 , or a fragment or portion thereof.

6. The antibody of claim 5, wherein the antibody is a Fab or F(ab′)₂ fragment of T7E 10, wherein T7D10binds specifically to a test antigen, and wherein the Fab or F(ab′)₂ fragment of T7E10 binds specifically to the same test antigen.

7. The antibody of claim 5, wherein the antibody is a scFv comprising at least a portion of the variable domain of the heavy chain of T7E10 or at least a portion of the variable domain of the light chain of T7E10or both, wherein T7E10binds specifically to a test antigen, and wherein the scFv binds specifically to the same test antigen.

8. The antibody of claim 2, wherein the antibody comprises T1G11 produced by a hybridoma cell line deposited as ATCC Accession No. PTA-9724 , or a fragment or portion thereof.

9. The antibody of claim 8, wherein the antibody is a Fab or F(ab′)₂ fragment of T1G11, wherein T1G11 binds specifically to a test antigen, and wherein the Fab or F(ab′)₂ fragment of T1G11 binds specifically to the same test antigen.

10. The antibody of claim 8, wherein the antibody is a scFv comprising at least a portion of the variable domain of the heavy chain of T1G11 or at least a portion of the variable domain of the light chain of T1G11 or both, wherein T1G11 binds specifically to a test antigen, and wherein the scFv binds specifically to the same test antigen.

11. The antibody of claim 2, wherein the antibody comprises F7B8 produced by a hybridoma cell line deposited as ATCC Accession No. PTA-9723 , or a fragment or portion thereof.

12. The antibody of claim 11, wherein the antibody is a Fab or F(ab′)₂ fragment of F7B8, wherein F7B8 binds specifically to a test antigen, and wherein the Fab or F(ab′)₂ fragment of F7B8 binds to the same test antigen.

13. The antibody of claim 11, wherein the antibody is a scFv comprising at least a portion of the variable domain of the heavy chain of F7B8 or at least a portion of the variable domain of the light chain of F7B8 or both, wherein F7B8 binds specifically to a test antigen, and wherein the scFv binds specifically to the same test antigen.

14. The antibody of claim 2, wherein the antibody comprises F1G11 produced by a hybridoma cell line deposited as ATCC Accession No. PTA-9725 , or a fragment or portion thereof.

15. The antibody of claim 14, wherein the antibody is a Fab or F(ab′)₂ fragment of F1G11, wherein F1G11 binds specifically to a test antigen, and wherein the Fab or F(ab′)₂ fragment of F1G11 binds specifically to the same test antigen.

16. The antibody of claim 14, wherein the antibody is a scFv comprising at least a portion of the variable domain of the heavy chain of F1G11 or at least a portion of the variable domain of the light chain of F1G11 or both, wherein F1G11 binds specifically to a test antigen, and wherein the scFv binds specifically to the same test antigen.

17. An antibody, comprising an iummunoglobulin polypeptide having a similar chemical structure to T7E10, wherein T7E10 is produced by a hybridoma cell line deposited as ATCC Accession No. PTA-9722 , wherein T7E10 binds specifically to a test antigen, and wherein the antibody having a similar chemical structure to T7E10 binds specifically to the same test antigen.

18. An antibody, comprising an iummunoglobulin polypeptide having a similar chemical structure to T1G11, wherein T1G11 is produced by a hybridoma cell line deposited as ATCC Accession No. PTA-9724 , wherein T1G11 binds specifically to a test antigen, and wherein the antibody having a similar chemical structure to T1G11 binds specifically to the same test antigen.

19. A kit, comprising the antibody of claim 2 and at least one test reagent.

20. The kit of claim 19, wherein the at least one test reagent comprises at least one solution.

21. A kit, comprising the antibody of claim 2 and at least one item of test equipment.

22. The kit of claim 21, wherein the at least one item of test equipment comprises at least one solid phase material.

23. The kit of claim 22, wherein the antibody is immobilized on or to the at least one solid phase material.

24. A kit, comprising two or more antibodies selected from the group consisting of T7E10T1G11, F7B8, and F1G11 produced by two or more hybridoma cell lines deposited as ATCC Accession Nos. PTA-9722, PTA-9724, PTA-9723, and PTA-9725, or fragments or portions thereof.

25. The kit of claim 24, wherein the two or more antibodies comprise T7E10 and F7B8 produced by hybridoma cell lines deposited as ATCC Accession Nos. PTA-9722 and PTA-9723 , respectively.

26. The kit of claim 25, further comprising at least one solid phase material.

27. The kit of claim 26, wherein F7B8 is immobilized on or to the surface of the at least one solid phase material.

28. A method, comprising the following steps:
(a) combining a primary detection antibody and the contents of a test sample; and
(b) determining whether a test antigen is bound by the primary detection antibody, wherein the test antigen is from a Pangasius species, wherein the primary detection antibody specifically binds to at least one epitope on the test antigen, and wherein the at least one epitope of the test antigen is not present in a non-Pangasius species.

29. The method of claim 28, wherein the Pangasius species is tra or basa.

30. The method of claim 28, wherein the test antigen is thermostable.

31. The method of claim 28, wherein the test sample comprises an extract from a food or agricultural product.

32. The method of claim 28, wherein the primary detection antibody comprises one or more of T7E10, F1G11, T1G11, or F7B8 produced by hybridoma cell lines deposited as ATCC Accession Nos. PTA-9722, PTA-9725, PTA-9724, and PTA-9723, respectively, or a fragment or portion thereof.

33. The method of claim 28, wherein the primary detection antibody is linked to an assayable tag or label.

34. The method of claim 28, wherein the determining step (b) comprises detecting the presence or amount of the assayable tag or label.

35. The method of claim 28, comprising the further step (c) of immobilizing at least a portion of the contents of the test sample on or to a solid phase material prior to step (a).

36. The method of claim 35, wherein the solid phase material is selected from the group consisting of a microtiter plate, magnetic beads, non-magnetic beads, a nitrocellulose membrane, a nylon membrane, a plastic dipstick, a glass dipstick, and paper.

37. The method of claim 35, wherein any contents of the test sample that are not immobilized on or to the solid phase material during step (c) are removed prior to step (a).

38. The method of claim 35, wherein any primary detection antibody that is not bound during step (a) to the test antigen immobilized on or to the solid phase material is removed prior to step (b).

39. The method of claim 35, wherein the determining step (b) comprises detecting changes in the electrochemical environment of the solid phase material.

40. The method of claim 35, wherein the determining step (b) comprises detecting changes in the refractive index of incident light near the surface of the solid phase material.

41. The method of claim 35, wherein the solid phase material comprises a piezoelectric crystal, and wherein the determining step (b) comprises detecting changes in the resonant frequency of the piezoelectric crystal.

42. The method of claim 35, comprising the further step (d) of adding a secondary detection antibody that binds to the primary detection antibody prior to step (b).
